(19) United States
(12) Patent Application Publication
(10) Pub. No.: US 2002/0192217 A1
(43) Pub. Date: Dec. 19, 2002

(54) Methods for regulation of immune responses to conditions involving mediator-induced pathology

(76) Inventors: Thierry Calandra, Lausanne (CH); Thierry Roger, Lausanne (CH); Michel P. Glauser, Lausanne (CH)

Correspondence Address: MINTZ, LEVIN, COHN, FERRIS,, GLOVSKY and POPEO, P.C., One Financial Center, Boston, MA, 02111, US

(21) Appl. No.: 10/094,732

(22) Filed: Mar. 7, 2002

Related U.S. Application Data
(60) Provisional application No. 60/274,004, filed on Mar. 7, 2001.

(51) Int. Cl. A61K 39/395; A61K 48/00

(52) U.S. Cl. 424/145.1; 514/44

(57) ABSTRACT

The present invention relates to methods for inhibiting the release and/or biological activity of the cytokine macrophage migration inhibitory factor (MIF). In particular, the invention relates to the uses of such methods for the treatment of various conditions involving mediator-induced diseases or pathology, which include, but are not limited to sepsis, severe sepsis, septic shock, inflammation, graft versus host disease, and/or autoimmune diseases.

### RELATED APPLICATIONS

This application claims the benefit of U.S. Ser. No. 60/274,004, filed Mar. 7, 2001, which is incorporated herein by reference in its entirety.

### FIELD OF THE INVENTION

The present invention relates generally to methods for inhibiting the release and/or biological activity of migration inhibitory factor (MIF). In particular, the invention relates to the use of such methods for the treatment of various conditions involving a mediator-induced diseases or pathology.

### BACKGROUND OF THE INVENTION

Septic shock is a multifaceted pathological condition characterized most prominently by deleterious hemodynamic changes and coagulopathy leading to multiple organ failure and, often, to death. The altered physiological mechanisms underlying septic shock syndrome, and the cellular means by which these changes are induced and controlled, are not yet known in precise detail. It is now generally accepted that septic shock reflects the individual, combined, and concerted effects of a large number of endogenous, host-derived mediator molecules. These mediators are produced in response to initiating stimuli that indicate the host has been invaded. Many classes of such peptide mediators which were first recognized as white blood cell products has come to be known as the cytokines. As mediators of toxic effects and pathological alterations in host homeostasis, these endogenous factors represent potentially attractive therapeutic targets, and septic shock remains a potentially lethal mediator-induced disease or pathology/clinical complication against which there is no generally effective therapeutic approach.

These host-derived mediators include many now well-recognized inflammatory cytokines and classical endocrine hormones, in addition to a number of other endogenous factors such as leukotrienes and platelet activating factor, among many others. The full cast of participants and their interrelated roles in the host response remains incompletely appreciated.

Those mediators that appear earlier in an invaded host are thought to trigger the release of the later appearing factors. Additionally, many endogenous mediators not only exert direct effector functions at their target tissues, but also prime local and remote tissues for subsequent responses to other mediators. This interacting network of host factors has been termed the “cytokine cascade.” This term is meant to indicate the rapid extension and amplification of the host response in such a way that only one or a few initiating stimuli trigger the eventual release and participation of scores of host mediators. Although a number of features of the host response are thought to assist in fighting off invasion, an overly robust or poorly modulated endogenous response can rapidly accelerate to produce such profound alterations in host homeostasis at cellular, tissue, and systemic levels that death may ensue within hours.

Among the interacting factors that comprise the cytokine cascade, is the important cytokine known as tumor necrosis factor alpha (TNF-α). TNF-α is one of the first cytokines to appear in the circulation after LPS challenge. The hemodynamic and metabolic alterations that result from experimental administration of TNF-α closely resemble those that have been observed in endotoxemia and septic shock. For example, in animal models TNF-α is the only host factor, which can initiate by itself a lethal syndrome that mimics septic shock. In this respect, TNF-α can be considered a mediator of septic shock.

Other cytokines participate in the host response to LPS but appear later in the circulation. Certain interleukins (IL-1, IL-6 and IL-8), which appear in serum more than 2 hours after LPS challenge, and interferon gamma (IFN γ), which appears after 6 hours, are thought to play a significant role in septic shock and can be shown to contribute to lethality in certain disease models or under experimental conditions of endotoxemia. Antagonism of the effects of specific interleukins and interferons has been shown to confer a significant protective effect under certain conditions. Nevertheless, except for IL-1, none of the other factors can itself induce a full-blown septic shock-like effect in otherwise healthy individuals, and none of these other cytokines appears to play as central and critical role in septic shock as TNF-α.

In view of the foregoing, TNF-α stands as an ideal target for the treatment of septic shock. Unfortunately, temporal characteristics of the endogenous TNF-α response suggest a significant practical limitation for this potential therapy. TNF-α is one of the earliest elicited mediators to appear in acute disease, rapidly peaks after bolus endotoxin challenge (30-90 minutes), and diminishes just as promptly. Thus, TNF-α antagonists such as anti-TNF-α antibodies would ideally need to be present at this time to be effective inhibitors of the TNF-α activity. Since this therapeutic window is so short and occurs so early in the cycle, the timely delivery of anti-TNF-α-based therapeutics may be very difficult to achieve clinically.

The pituitary gland may produce factors that inhibit endotoxin-induced TNF-α and IL-1 production, and thus may serve as a source for potentially protective factors that may be used to treat shock and/or other inflammatory responses. See Suzuki et al., 1986, Am. J. Physiol. 250: E470-E474; Sternberg et al., 1989, Proc. Natl. Acad. Sci. USA 86: 2374-2378; Zuckerman et al., 1989, Eur. J. Immunol. 19: 301-305; Edwards III et al., 1991a, Endocrinol. 128: 989-996; Edwards III et al., 1991b, Proc. Natl. Acad. Sci. USA 88: 2274-2277, Silverstein et al., 1991, J. Exp. Med. 173:357-365. In these studies, hypophysectomized mice (i.e, animals that have had their pituitary glands surgically removed) exhibited a marked increased sensitivity to LPS injection relative to sham-operated control mice. In fact, the LPS LD₁₀₀ for control mice was approximately 1-2 logs higher than that determined for the hypophysectomized mice, suggesting that the pituitary gland produces one or more factors that may act to increase the host animal's ability to resist endotoxin challenge. Some of these studies implicate the involvement of ACTH and adrenocorticosteroids (e.g., Edwards III et al., 1991a and 1991b, supra). Other data suggest the existence of other protective factors derived from the pituitary.

### SUMMARY OF THE INVENTION

In one aspect, the invention involves a method for treating conditions involving a mediator-induced diseases or pathology condition involving administering a therapeutically effective amount of an agent that decreases the endogenous amount of intracellular or extracellular MIF in a patient. As used herein, a “decrease in the amount of intracellular or extracellular MIF” refers to a decrease in the amount of MIF present in unstimulated macrophages. By “therapeutically effective amount” is meant an amount sufficient to produce a therapeutic effect in a patient in need thereof. A “condition involving a mediator-induced diseases or pathology” refers to, for example, endotoxin-induced septic shock, endotoxin-induced toxic shock, sepsis, severe sepsis, septic shock caused by Gram-negative bacteria, bacterial infections, shock, inflammatory diseases, graft versus host disease, autoimmune diseases, acute respiratory distress syndrome, granulomatous diseases, chronic infections, transplant rejection, acute respiratory asthma, viral infections, parasitic infections, fungal infections, and/or trauma. Those skilled in the art will recognize that cytokines are part of a larger group of molecules that have been designated “mediators”. Any other molecules that belong to the class of molecules known as “mediators” are also included as part of the instant invention.

In various embodiments of the invention, an agent that decreases the endogenous amount of intracellular or extracellular MIF in a patient may include an antisense nucleic acid, a small molecule inhibitory compound, a peptide mimetic, an agent that down-regulates MIF expression, an antibody, and an inhibitor of MIF activity. As used herein, a “peptide mimetic” includes any peptide analogs in which one or more peptide bonds have been replaced with an alternative type of covalent bond (a “peptide mimetic”), which is not susceptible to cleavage by peptidases. Where proteolytic degradation of the peptides following injection into the subject is a problem, replacement of a particularly sensitive peptide bond with a noncleavable peptide mimetic will make the resulting peptide more stable and thus more useful as a therapeutic. Such mimetics, and methods of incorporating them into peptides, are well known in the art. Similarly, the replacement of an L-amino acid residue with a D-amino acid is a standard way of rendering the peptide less sensitive to proteolysis. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. Blocking the charged amino- and carboxy-termini of peptides has the additional benefit of enhancing passage of a peptide through the hydrophobic cellular membrane and into the cell.

Moreover, an “agent that down-regulates MIF expression” refers to any agent that functions to decrease or down-regulate the expression of the MIF protein from the Mif gene. Likewise, an “inhibitor of MIF activity” is any agent that inhibits the activity of the MIF protein. One skilled in the art will recognize that any suitable “small molecule inhibitory compound” that decreases the endogenous amount of intracellular or extracellular MIF can be employed in the methods of the present invention.

In another embodiment, the invention is concerned with diseases or disorders associated with a mediator-induced diseases or pathology that are the result of infectious agents. By “infectious agents” is meant any pathogen that may cause or contribute to a mediator-induced diseases or pathology. By way of non-limiting example, infectious agents may include microbial toxins (including Gram-negative bacterial endotoxin, Gram-positive bacterial endotoxins, toxins of other microbial agents, etc.) and/or cell wall fragments of microbial pathogens (including peptidoglycan, lipoteichoic acids, etc.). Those skilled in the art will recognize that pathogens will also be included as “infectious agents”. In one embodiment, the invention is concerned with treating bacterial infections caused by Gram-negative bacteria.

In a further aspect, the invention concerns a method for treating a disease such as graft versus host disease, acute respiratory distress syndrome, granulomatous disease, transplant rejection, cachexia, parasitic infections, fungal infections, trauma, and bacterial infections by administering a therapeutically effective amount of an agent that decreases the endogenous amount of intracellular or extracellular MIF to a patient suffering from the disease.

In another aspect, the invention is concerned with a method for treating an inflammatory or infectiouscondition or disease by administering a therapeutically effective amount of an agent that decreases the endogenous amount of intracellular or extracellular MIF to a patient suffering from the inflammatory condition or disease. One skilled in the art will recognize that the term “an inflammatory or infectious condition or disease” includes, but is not limited to, for example, autoimmune or inflammatory diseases such as multiple sclerosis, inflammatory bowel disease, insulin dependent diabetes mellitus, and rheumatoid arthritis, trauma, chemotherapy reactions, transplant rejections the generalized Schwarzmann reaction, system inflammatory response syndrome, sepsis, severe sepsis, or septic shock.

In still another aspect, the invention involves a method for treating an individual having a disease caused by mediator-induced diseases or pathology. In this aspect, the method involves administering to a patient an effective amount of an agent that decreases the endogenous amount of intracellular or extracellular MIF and a pharmaceutically acceptable carrier or diluent. In one embodiment, this aspect additionally involves administering a therapeutic steroid to the patient. By way of non-limiting example, therapeutic steroids may include, for example, glucocorticoids, dexamethasone, prednisone, prednisalone, and betamethasone.

In a further aspect, the invention concerns a method for enhancing the anti-inflammatory activity of a therapeutic steroid or reducing the toxic side effects of a therapeutic steroid by administering a therapeutically effective amount of an agent that decreases the endogenous amount of intracellular or extracellular MIF in an individual in need of such treatment.

In another aspect, the invention involves a method for treating a condition involving a mediator-induced diseases or pathology by administering to a patient an effective amount of an agent that down-regulates a Toll-like receptor (TLR). In one embodiment, the agent that down-regulates the Toll-like receptor does so by decreasing the endogenous amount of intracellular or extracellular MIF. Examples of such agents may include antisense nucleic acids, small molecule inhibitory compounds, peptide mimetics, agents that down-regulate Toll-like receptor expression, agents that down-regulate MIF expression, inhibitors of Toll-like receptor activity, an antibody, and inhibitors of MIF activity. In another embodiment, the Toll-like receptor may be Toll-like receptor 4 (TLR4). In this embodiment, the agent that down-regulated the Toll-like receptor 4 does so by decreasing the endogenous amount of intracellular or extracellular MIF. In various other embodiments, the Toll-like receptor may be any of the Toll-like receptors. For example, the Toll-like receptor may be Toll-like receptor 1 (TLR1), Toll-like receptor 2 (TLR2), Toll-like receptor 3 (TLR3), Toll-like receptor 4 (TLR4), Toll-like receptor 5 (TLR5), Toll-like receptor 6 (TLR6), Toll-like receptor 7 (TLR7), Toll-like receptor 8 (TLR8), Toll-like receptor 9 (TLR9), or Toll-like receptor 10 (TLR10).

In yet another aspect, the invention involves a method for treating a condition involving mediator-induced diseases or pathology by administering a therapeutically effective amount of an anti-MIF antibody or antigen binding fragment thereof given alone or in combination with an agent that decreases the endogenous amount of intracellular or extracellular MIF.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description and from the claims.

### BRIEF DESCRIPTION OF THE DRAWINGS

FIG. 1 shows the hyporesponsiveness of antisense mouse MIF macrophages to activation by LPS and Gram-negative bacteria. Panel a shows the Western blot analysis of intracellular MIF content of wild-type RAW 264.7 macrophages stably transfected with an empty plasmid (pBK 1.1) or with an antisense MIF cDNA plasmid (AS 2.8 and AS 2.23). 10 μl of cell lysate (equivalent to 5×10⁴ cells) were electrophoresed through 15% gels, transferred to nitrocellulose membranes, and analyzed with anti-MIF polyclonal antibody. 50 ng of recombinant MIF (rMIF) was electrophoresed and transferred as a standard. Panesl b-d show TNF-α production pBK 1.1 (black bars), AS 2.8 (white bars) and AS 2.23 (hatched bars) in macrophages stimulated with LPS, 10⁸ CFU of heat-killed bacteria, or PMA plus inomycin (PMA+iono). Data present are the mean±s.d. of triplicat samples from one experiment and are representative of 3-9 independent experiments. Panel e shows a Northern blot of TNF-α and β-actin mRNA in pBK 1.1, AS 2.8, and AS 2.23 macrophages stimulated for 2 h with LPS. Data are representative of four separate experiments. See Roger et al., Nature 414:920-24 (2001).

FIG. 2 shows that MIF regulates macrophage responses to LPS by modulating expression of TLR-4. Panel a shows a Northern blot of basal TLR4, TLR2, MD-2 and GAPDH mRNA in pBK 1.1, AS 2,8, and AS 2.23 RAW 264.7 macrophages. Panel b shows TLR-4-MD-2 expression on pBK 1.1, AS 2.8, and AS 2.23 macrophages analyzed by flow cytometry. Backgrown staining is shown in grey. Panel c shows that transfection of antisense MIF macrophages with mouse TLR4-pCMV restores responsiveness to LPS, as assessed by measuring nF-κB-driven luciferase activities 6 h after stimulation with (black bars) or without (white bars) LPS (100 ng ml⁻¹). Panel d shows that dominant-negative TLR4 abolished TNF-α production by antisense MIF macrophages transfected with TLR4 and stimulated for 4 h with (black bars) or without (white bars) LPS (100 ng ml⁻¹). Panels e and f show that raising the MIF content of antisense MIF macrophages restores TLR4 expression and responsiveness to LPS. pBK1.1, AS 2.8, and AS 2.23 macrophages were transfected with an empty plasmid (−) or with a MIF expression plasmid (+). Panel e is a Western blot of intracellular MIF and northern blots of TLR4 and GADPH mRNA. Panel f shows TNF-α production by AS 2.8 and AS 2.23 stimulated for 4 h with (black bars) or without (white bars) LPS (100 ng ml⁻¹).

FIG. 3 demonstrates that decreased basal TLR4 expression and LPS-induced TNF-α production by MIF knockout (MIF^{-/-}) macrophages. Panel a shows Northern blots of basal MIF, TLR4, MD-2, and GAPDH mRNA of MIF^{+/+} and MIF^{-/-} macrophages. Panel b shows the results of flow cytometry analysis of TLR4-MD-2 expression of thioglycollate-elicited peritoneal macrophages isolated from MIF^{+/+} (n=25) and MIF^{-/-} (n=27) mice (P=0.002). As used herein, “m.f.i.” refers to the mean fluorescence intensity. The box plots show the 10^{th}, 25^{th}, 50^{th}, 75^{th}, and 90^{th} percentiles, respectively. Panel c shows representative histograms of TLR4-MD-2 expression in MIF^{+/+} and MIF^{-/-} macrophages. Background staining is shown in grey. Panels d and e show the TNF-α production by MIF^{+/+} (black bars) and MIF^{-/-} (white bars) macrophages stimulated with LPS, 10⁸ colony forming units (c.f.u.) of heat-killed bacteria, peptidoglycan (100 μg ml⁻¹) or zymosan (2×10⁶). Data shown are the mean±s.d. of two independent experiments with cells isolated from a total of four mice per group.

FIG. 4 shows that basal TLR4 promoter activity and PU.1 DNA-biding activity are reduced in antisense MIF RAW 264.7 macrophages. Panel a shows the activity of deletion constructs of the mouse Tlr4 promoter cloned into a luciferase reporter plasmid and transfected into RAW 264.7 macrophages. Panel b shows Tlr4 promoter activity of pBK 1.1, AS 2.8, and AS 2.23 macrophages transfected with the −518 TLR4 luciferase construct. Panel c shows PU.1 activity of pBK1.1, AS 2.8, and AS 2.23 macrophages transfected with a trimeric MHC-derived PU.1 luciferase reporter construct. Data shown in panels a-c are the mean±s.d. of 2-5 independent experiments. Panel d shows the electrophoretic mobility shift assay showning reduced basal PU.1 DNA-binding activity in antisense MIF macrophages. PU.1 specific complexes are marked with arrowheads.

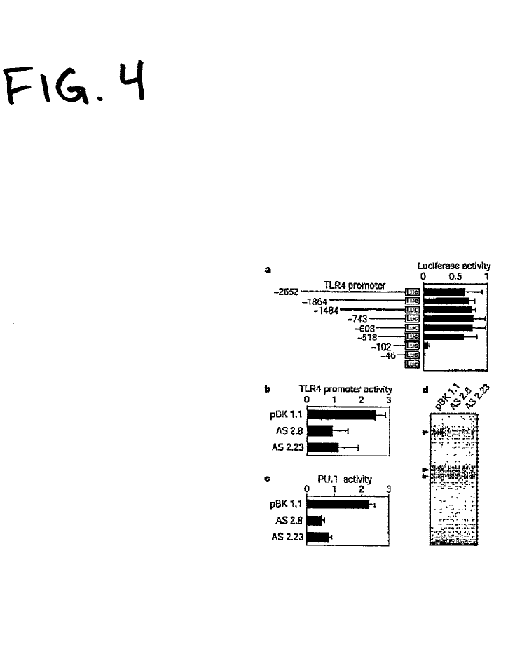

FIG. 5 shows reduced NF-κB activity and TNF-α gene activation in antisense MIF macrophages. In Panels a, b, and c, LPS-induced NF-κB DNA binding activity is decreased, but not delayed, in antisense MIF macrophages. Time-course (Panel a) and dose-response (Panel b) studies of NF-κB DNA binding activity of control (pBK 1.1; black bars) and antisense MIF (AS 2.8 and AS 2.23; white and hatched bars) RAW 264.7 macrophages stimulated with LPS (100 ng/ml in Panel a for 0-60 min (Panel a) or 30 min (Panel b). Cells were transfected with a multimeric NF-κB luciferase vector and Renilla pRL-TK vector and incubated for 6 h with LPS. Data are expressed as a relative luciferase activity (n=3).

FIG. 6 shows CD14 expression (left panel) and FITC-LPS binding (right panel) analyzed by flow cytometry. The grey area shows background fluorescence. Mean±SD of fold-increase over background flourescense of pBK 1.1, AS 2.8, and AS 2.23 was 5.2±1.2, 9±5.4 and 8.8±3.3 for CD14 expression (n=7, P-0.46), and 8.2±3.0, 11.2±4.5 and 7.9+0.1 for LPS binding (N=2, P=0.47), respectively.

FIG. 7 shows the results observed when antisense MIF macrophages were transfected with 0.05 μg of the Renille pRL-TK vector, 2 μg of a multimeric NF-κB-luciferase vector, 0.4 μg of an expression plasmid encoding mouse TLR4 (mTLR4) and 2 μg of an expression plasmid encoding a dominant negative mTLR4 (dn mTLR4). Cells were incubated for 6 h with LPS. Data are expressed as relative luciferase activity.

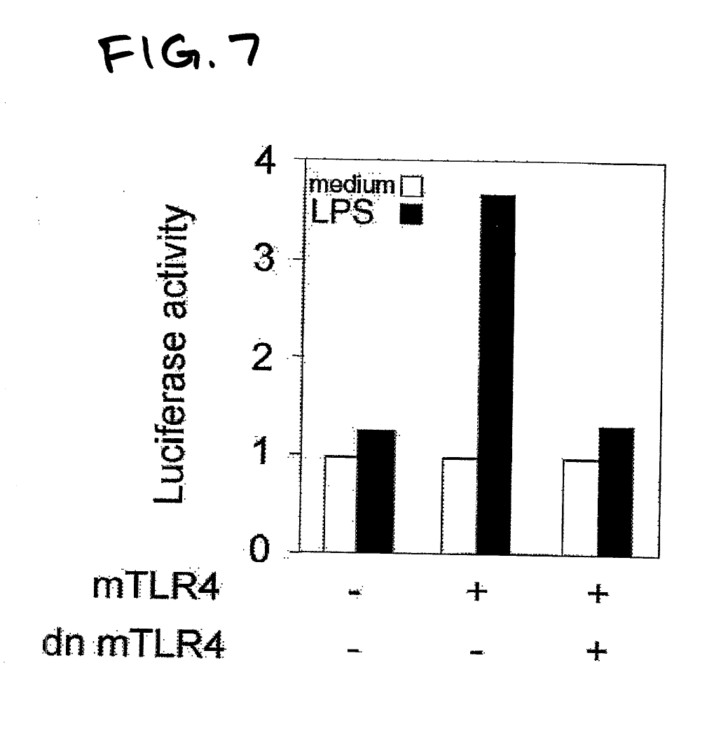

FIG. 8 demonstrates that TNF-α. mRNA decay is not increased in antisense MIF macrophages stimulated with LPS. Panel A shows the results of Northern blot analysis of TNF-α mRNA in control and antisense MIF macrophages. Parallel cultures of control and antisense MIF RAW 264.7 macrophages (1×10⁶ cells per well in 6-well tissue culture plates) were stimulated for 2 h with LPS (100 ng/ml), and then incubated with actinomycin D (10 μg/ml) (Roche Diagnostics) for an additional 0, 15, 30 or 60 min. Total RNA was extracted with Trizol™ (Gibco BRL), run through an agarose-formaldehyde gel, blotted on nylon membrane, and hybridized with TNF-α and β-actin probes. Panel b shows the time-course of TNF-α. mRNA decay in control pBK 1.1 macrophages (closed circles) and antisense MIF macrophages (AS 2.8: open circles; AS 2.23: open triangles). TNF-α mRNA levels were normalized for β-actin mRNA levels and expressed as the percentage of the signal present at time 0 (i.e. before addition of actinomycin D). Data are mean±SD of three independent experiments.

FIG. 9 shows that human TLR4, but not TLR1, TLR2 and TLR3 restores NF-κB activity of antisense MIF macrophages. Control (pBK 1.1) and antisense MIF (AS 2.8 and AS 2.23) RAW 264.7 macrophages were transiently co-transfected with 0.04 μg of the Renilla pRL-TK vector, 2 μg of a multimeric NF-κB pGL2 luciferase reporter vector and with 0.4 μg of expression plasmids encoding either human TLR1, or TLR2, or TLR3 or TLR4, or the corresponding empty pFlag-CMV vector (pCMV). Twenty-four h after transfection, cells were incubated for 6 h with (black bars) or without (white bars) LPS (100 ng/ml). Luciferase and Reni luciferase activities of cell lysates were measured as described in the Examples and the results expressed as relative luciferase activity.

### DETAILED DESCRIPTION OF THE INVENTION

Murine macrophage migration inhibitory factor (MIF) has been identified as an LPS-induced pituitary protein. (Roger et al., Nature 414:920-24 (2001) (incorporated herein by reference). MIF may be one of the protective factors capable of counteracting the adverse effects of mediators and cytokines in endotoxemia and sepsis.

Macrophages are pivotal effector cells of the innate immune system that play an essential role in the recognition and elimination of invasive microbial pathogens. See Hoffmann et al., Science 284:1313-18 (1999); Aderem et al., Annu. Rev. Immunol. 17:593-23 (1999). When macrophages are activated by the binding of microbial products to pathogen-recognition receptors, macrophages release a broad array of cytokines that orchestrate the innate and adaptive immune responses of the host. See Dinarello, Chest 118:503-08 (2000). MIF has been found to be an important mediator of sepsis, and, unlike other cytokines, it is constitutively expressed by unstimulated monocytes and macrophages. See Calandra et al., J. Exp. Med. 179:1895-902 (1994).

Compared to wild-type cells, MIF-deficient macrophages are hyporesponsive to endotoxin and Gram-negative bacteria, as shown by a profound reduction of NF-κB activity and TNF-α production. Recent studies indicated that this impaired response was due to a down-regulation of Toll-like receptor 4 (TLR4), which is the signal-transducing molecule of the LPS receptor complex. See Roger et al., Nature 414:920-24 (2001).

Although MIF was first described over 25 years ago as a T cell product that inhibits the random migration of guinea pig macrophages (Bloom & Bennett, 1966, Science 153: 80-82; David, 1966, Proc. Natl. Acad. Sci. USA 56: 72-77), the precise role of MIF in either local or systemic inflammatory responses has remained largely undefined. MIF has been reported to be associated with delayed-type hypersensitivity reactions (Bloom & Bennett, 1966, supra; David, 1966, supra), to be produced by lectin-activated T-cells (Weiser et al., 1981, J. Immunol. 126: 1958-1962), and to enhance macrophage adherence, phagocytosis and tumoricidal activity (Nathan et al., 1973, J. Exp. Med. 137: 275-288; Nathan et al., 1971, J. Exp. Med. 133: 1356-1376; Churchill et al., 1975, J. Immunol. 115: 781-785). However, many of these studies used mixed culture supernatants that were later shown to contain other cytokines including IFN γ and IL-4 which also have migration inhibitory activity (McInnes & Rennick, 1988, J. Exp. Med. 167: 598-611; Thurman et al., 1985, J. Immunol. 134: 305-309).

Recombinant human MIF was originally cloned from human T cells (Weiser et al., 1989, Proc. Natl. Acad. Sci. USA 86: 7522-7526), and has been shown to activate blood-derived macrophages to kill intracellular parasites and tumor cells in vitro, to stimulate IL-1β and TNF-α expression, and to induce nitric oxide synthesis. See Weiser et al., 1991, J. Immunol. 147: 2006-201 1; Pozzi et al., 1992, Cellular Immunol. 145: 372-379; Weiser et al., 1992, Proc. Natl. Acad. Sci. USA 89:8049-8052; Cunha et al., 1993, J. Immunol. 150:1908-1912.

The mouse Mif gene has been mapped to chromosome 10 and lies between the Bcr and S100b loci. See Mitchell et al., J. Immunol. 154:3863-70 (1995); Kozak et al. Genomics 27:405-11 (1995). Human Mif has been mapped to a region of chromosome 22 (22q11.2) that is known to be in syntenic conservation with the section of mouse chromosome 10 that contains the Mif gene. See Budarf et al., Genomics 39(2):235-36 (1997). The predicted coding sequence of the Mif gene is identical to sequences of the Mif cDNA of the human eye lens (see Wistow et al., PNAS 90:1272-75 (1993)) and the Jurkat human T-cell line (see Bernhagen et al., Biochemistry 33:14144-55 (1994)). Moreover, the mouse and human MIF proteins share 90% amino acid identity. In fact, MIF proteins exhibit more than 80% homology across species such as mouse, rat, gerbil, chicken, calf, and human.

MIF is identical to a protein known as glycosylation-inhibiting factor (GIF), which has been shown to suppress IgE synthesis and be associated with antigen-specific suppressor activity. See Ishizaka et al., Adv. Immunol. 74:1-60 (2000). MIF also has 33% amino acid sequence homology to D-dopachrome tautomerase. See Sugimoto et al., Biochemistry 38(11):3268-79 (1999). However, no significant sequence homology between MIF and other cytokines has been determined, which indicates that MIF does not belong to any known cytokine superfamily. Additionally, MIF lacks a classical N-terminal leader sequence. Thus it is likely released from cells by a nonconventional protein secretion pathway.

MIF has been found to enhance a broad-spectrum of macrophage functions, including adherence, phagocytosis, spreading, and tumoricidal activity (reviewed in Calandra et al., Critical Reviews in Immunology 17:77-88 (1997)). However, the biological activities ascribed to MIF remained uncertain, as cytokines such IFNγ and IL-4 were also observed to inhibit macrophage migration. Many human organs, including the kidney, brain, and liver, express high MIF mRNA levels at baseline. As detailed in Table 1, MIF is found in a wide variety of tissues. See Baugh et al., Crit. Care Med. 30(1) (Suppl):S27-S35 (2002).

TABLE 1
Tissue Distribution and Cellular Sources of MIF

| | |
|---|---|
| Immune system | Monocytes/macrophages, T cells, B cells |
| | Eosinophils, mast cells, basophils |
| | Neutrophils |
| | Spleen (white pulp, red pulp) |
| Endocrine system | Pituitary gland (corticotrophic cells) |
| | Adrenal cortex (zona glomerulosa, zona fasciculata) |
| | Pancreas (β cells of the islet) |
| Brain | Cortex (neurons), hypothalamus, cerebellum (neurons), |
| | hippocampus, pons, glial cells, ependyma, astrocytes |
| Kidney | Epithelial cells (proximal tubules, collecting ducts, glomeruli, |
| | Bowman's capsule), mesangial cells, endothelium, central veins, |
| | Kuppfer cells |
| Lung | Aveolar macrophages, epithelial cells (bronchi) |
| Liver | Kuppfer cells, hepatocytes surrounding central veins, endothelium |
| | (central venules) |
| Gastrointestinal tract | Epithelial cells (esophagus, stomach, small and large intestines) |
| | Neurons (myenteric and submucosal plexi) |
| Skin | Keratinocytes, sebaceous gland, hair follicle (outer root sheet) |
| | endothelial cells, fibroblasts |
| Eye | Lens, epithelial cells (cornea, iris, ciliary body) |
| | endothelial cells, retina (epithelium, Muller cells, astrocytes) |
| Testis | Leydig cells |
| Prostate | Epithelial cells |
| Ovary | Granulosa cells of follicles |
| Bone | Osteoblasts |
| Fat tissue | Adipocytes |
| Vasculature | Endothelial cells |

In 1994, cells of the monocyte/macrophage lineage were identified to be an abundant source of MIF. See Calandra et al., J. Exp. Med. 179:1895-1902 (1994). Unlike other cytokines, high baseline levels of MIF mRNA and MIF protein are present in resting macrophages. Macrophages release MIF when stimulated with microbial products (e.g. LPS, toxic shock syndrome toxin-1 [TSST-1], streptococcal pyrogenic exotoxin A [SPEA] and malaria pigment), bacteria (Gram-negative and Gram-positive, M. tuberculosis) and cytokines (TNF-α and IFN γ). See id.; Calandra et al., PNAS 95:11383-88 (1998); Calandra et al., Nat. Med. 6(2):164-70 (2000); and Martiney et al., Infect. Immun. 68(4):2259-67 (2000).

MIF induces TNF-α secretion by macrophages and synergizes with IFN γ to augment nitric oxide production. Thus, MIF works in concert with these two cytokines to intensify pro-inflammatory responses of macrophages. See Calandra et al., Critical Reviews in Immunology 17:77-88 (1997); Bernhagen et al., Biochemistry 33:14144-14155 (1994). Unexpectedly, glucocorticoid hormones were found to induce, rather than to inhibit MIF secretion. See Calandra et al., Nature 377:68-71 (1995).

Pre-formed MIF protein has also been detected in resting T-cells. See Bacher et al., PNAS 93:7849-54 (1996). Previous studies have suggested a role for MIF in activation of B-cells, and anti-MIF antibodies have been shown to inhibit antibody production by B-cells. See id Moreover, experimental results suggest a role for MIF in the control of cell cycle progression and apoptosis in B-cells.

Innate immune cells (including neutrophils, macrophages, NK cells), along with complement, are the first line of host defenses against invading microorganisms and as such are a critical element of the host defense system. MIF protein is expressed in neutrophils, however in much lower quantities than in either macrophages or T-cells. See Swope et al., EMBO Journal 17(13):3534-3541 (1998). It has thus been suggested that MIF might exert a priming effect on neutrophils. See id.

Unstimulated eosinophils, like monocytes, macrophages, T-cells and B-cells, have also been described as an abundant cellular source of MIF. See Rossi et al., J. Clin. Invest. 101(12):2869-74 (1998). Along with monocytes, macrophages and neutrophils, natural killer (NK) cells also contribute to innate immune responses.

In studies that examined the mechanisms sustaining immune privilege in the anterior chamber of the rabbit eye, a factor sharing more than 90% homology with residues 95-108 of human MIF was shown to inhibit the NK-mediated lysis of corneal endothelial cells. See Apte et al., J. Immunol. 160(12):5693-5696 (1998). It is also noteworthy that recombinant mouse MIF inhibited NK activity and neutralizing anti-MIF restored it. However, MIF was not directly toxic to NK cells. Rather, MIF exerted its effects by interfering with the release of cytolytic perforin granules from NK cells. It is unclear whether NK cells express MIF protein.

Some properties and biological activities of MIF are summarized in Table 2. See Baugh et al., Crit. Care Med. 30(1) (Suppl) S27-S35 (2002) (incorporated herein by reference).

TABLE 2
Properties and Biological Activities of MIF

| | |
|---|---|
| In Vitro | Phagocytosis of particles |
| | Glucocorticoid counter-regulator |
| | Promotes nitric oxide (NO) and TNF-α release from |
| | macrophages |
| | Mediator of T-cell activation and antigen-specific immunity |
| | Promotes insulin release from pancreatic beta cells |
| | Suppression of inhibin release from Leydig cells |
| | Regulator of glycolysis |
| | Mitogen for fibroblasts and endothelial cells |
| | Promotes tumor cell proliferation |
| | Promotes endothelial cell proliferation |
| In Vivo | Disease progression/pathologies (experimental animal models) |
| | Endotoxemia and exotoxemia |
| | Delayed-type hypersensitivity reaction |
| | Antigen-dependent T-cell activation |
| | Collagen-induced arthritis |
| | Adjuvant-induced arthritis |
| | Glomerulonephritis |
| | Tumor growth and angiogenesis |

As noted, MIF plays a role in host defenses against infection. See Bemnhagen et al., Nature 365:756-59 (1993); Calandra et al., J. Exp. Med. 179:1895-902 (1994); Calandra et al., PNAS 95:11383-388 (1998); Calandra et al., Nat. Med. 6(2):164-70 (2000); Roger et al., Nature 414:920-24 (2001). MIF also is implicated in the immune responses induced by staphylococcal and streptococcal exotoxins. In addition to septic shock, MIF has been shown to be involved in the pathophysiology of malaria. Moreover, elevated concentrations of MIF have been detected in alveolar airspaces of patients with the adult respiratory distress syndrome (ARDS). See Donnelly et al., Nature Medicine 3(3):320-23 (1997). Other researchers have reported that patients with atopic dermatitis have increased expression of MIF in keratinocytes and high circulating concentrations of MIF. See Shimizu et al. Biochem Biophys Res Commun 240(1):173-78 (1997).

Additionally, anti-MIF antibodies also reduced inflammation in experimental models of glomerulonephritis and allograft rejection, confirming a role for MIF in the regulation of inflammatory responses. Moreover, those skilled in the art will recognize that inflammation is an essential component of host defenses against infectious agents.

MIF is a critical mediator of the host responses to a broad-spectrum of microorganisms or microbial products. See Bernhagen et al., Nature 365:756-59 (1993); Calandra et al., J. Exp. Med. 179:1895-1902 (1994); Bacher et al., Am. J. Pathol. 150(1):235-46 (1997); Calandra et al., PNAS 95:11383-88 (1998); Calandra et al., Nat. Med. 6(2):164-70 (2000); Calandra et al., Nature 377:68-71 (1995); Roger et al., Nature 414:920-24 (2001). MIF additionally plays a role in antigen-specific T-cell activation and in the development of a humoral antibody response. See Bacher et al., PNAS 93:7849-54 (1996).

During investigations of factors that may serve to modulate sepsis-induced inflammatory responses, MIF was re-discovered as a pituitary peptide. LPS-stimulated AtT-20 anterior pituitary cells were found to release copious amounts of a 12.5-kDa protein. See Bernhagen et al., Nature 365:756-59 (1993). Subsequent N-terminal sequencing of this protein revealed that it was the mouse homologue of human MIF and shared 90% identity with human MIF. Cloning and sequencing of an AtT-20 MIF cDNA confirmed these results. Additional experiments indicated that MIF was secreted from the pituitary gland and from the adrenal cortex in a hormone-like fashion after stimulation by LPS. See Calandra et al., J. Exp. Med. 179:1895-902 (1994); Bacher et al., Am. J. Pathol. 150(1):235-46 (1997).

In recent years, several comprehensive reviews of the biochemical and biological properties of MIF have been published. See, e.g., Calandra et al, Critical Reviews in Immunology 17:77-88 (1997); Bernhagen et al., J. Mol. Med. 76:151-161 (1998); Swope et al., Rev. Physiol. Biochem. Pharmacol. 139:1-32 (1999); Baugh et al., Crit. Care Med. 30(1) (Suppl.) S27-S35 (2002). MIF has also recently been shown to exert intracellular activity. See Kleemann et al., Nature 408:211-16 (2000).

Once secreted, MIF exerts potent pro-inflammatory activities that stimulate macrophages to release cytokines and that activate T-cells. See Calandra et al., J. Exp. Med. 179:1895-902 (1994); Bacher et al., PNAS 93:7849-54 (1996). Plasma levels of MIF are up-regulated in patients with severe sepsis and septic shock. See Calandra et al., Nat. Med. 6:164-70 (2000); Beishuizen et al., J. Clin. Endocrinol. Metab. 86:2811-16 (2001); Mitchell et al., Proc. Natl. Acad. Sci. USA 99(1):345-350 (2002) Researchers have shown that neutralization of MIF with anti-MIF antibodies or the deletion of the Mif gene reduces cytokine production and confers protection in experimental models of endotoxemia and Gram-negative septic shock. See Bernhagen et al, Nature 365:756-59 (1993); Calandra et al, Nature 377:68-71 (1995); Calandra et al., Nat. Med. 6:164-170 (2000); Bozza et al., J. Exp. Med. 189:341-46 (1999).

Inhibition of MIF activity, in accordance with the invention, may be accomplished in a number of ways, which include, but are not limited to, the use of MIF binding partners, i.e., factors that bind to MIF and neutralize its biological activity, such as neutralizing anti-MIF antibodies, soluble MIF receptors, MIF receptor fragments, and MIF receptor analogs; the use of MIF-receptor antagonists, such as anti-MIF-receptor antibodies, inactive MIF analogs that bind but do not activate the MIF-receptor, small molecules that inhibit MIF release or activity (see Senter et al., Proc. Natl. Acad. Sci. USA 99(1):144-49 (2002)), alter the normal configuration of MIF, or inhibit productive MIF/MIF-receptor binding; or the use of nucleotide sequences derived from MIF gene and/or MIF receptor gene, including coding, non-coding, and/or regulatory sequences to prevent or reduce MIF expression by, for example, antisense, ribozyme, and/or triple helix approaches. Inhibition may also be accomplished by administration of an agent that decreases the endogenous amount of intracellular or extracellular MIF, such as an antisense nucleic acid, a small molecule inhibitory compound, a peptide mimetic, an agent that down-regulates MIF expression, and an inhibitor of MIF activity. Any of the foregoing methods may be utilized individually or in combination to inhibit MIF release and/or activity in the treatment of the relevant conditions. Further, such treatment(s) may be combined with other therapies that (a) inhibit or antagonize initiators of mediator-induced diseases or pathology (e.g. anti-LPS antibody); (b) inhibit or antagonize toxic participants in the endogenous cytokine responses (e.g. anti-TNF-α, anti-IL-1, anti-IFN γ, or IL-1 receptor antagonist (“RA”); or (c) themselves inhibit or antagonize mediator-induced diseases or pathology (e.g. steroids, glucocorticoids, IL-10; activated protein C class of agents, anticoagulants).

The present invention involves compositions and methods that inhibit MIF release and/or activity in vivo, for the treatment of any conditions involving a mediator-induced diseases or pathology, which include but are not limited to shock, including endotoxin-induced sepsis, severe sepsis, and septic shock and exotoxin-induced sepsis, severe sepsis, septic shock, inflammation, graft versus host disease, autoimmune diseases, acute respiratory distress syndrome, granulomatous diseases, chronic infections, transplant rejection, cachexia, bacterial infections, viral infections, parasitic infections, fungal infections, and/or trauma. Also included are diseases mediated by microbial toxins including Gram-negative bacterial endotoxin, Gram-positive bacterial endotoxins, toxins of other microbial or infectious agents, and cell wall fragments of microbial pathogens such as peptidoglycan and lipoteichoic acids.

The inhibition of MIF activity in accordance with the invention may be accomplished in a number of ways, which may include, but are not limited to, the use of factors which bind to MIF and neutralize its biological activity; the use of MIF-receptor antagonists; the use of compounds that inhibit the release of MIF from cellular sources in the body; the use of agents that inhibit the activity of MIF (including antisense nucleic acid molecules, small molecule inhibitory compounds, peptide mimetics, agents that down-regulate MIF expression, antibodies, and other inhibitors of MIF activity), and the use of nucleotide sequences derived from MIF coding, non-coding, and/or regulatory sequences to prevent or reduce MIF expression. Any of the foregoing may be utilized individually or in combination to inhibit MIF activity in the treatment of the relevant conditions, and further, may be combined with any other anti-cytokine therapy (including steroid therapy), inhibitory cytokines, anti-sepsis agents, anti-inflammatory, immunomodulating agents, or any combination thereof.

MIF is identified herein as both a neuroendocrine mediator and a macrophage cytokine, and it plays an important role in host inflammatory responses to infection and tissue invasion. Upon release after a proinflammatory stimulus, macrophage-derived MIF may act together with TNF-α and other cytokines or mediators to coordinate local cellular responses against infection or tissue invasion. Pituitary-derived MIF, however, may serve to prime systemic immune responses once a localized inflammatory site fails to contain an invasive agent, or else act as a CNS-derived stress signal to activate the immune system against invasion. Thus, MIF may act in concert with hormones of the hypothalamus-pituitary-adrenal axis to modulate systemic inflammatory responses.

MIF plays a critical role in endotoxin-induced septic shock (caused by Gram-negative organisms), endotoxin-induced sepsis, severe sepsis, and septic shock and exotoxin-induced sepsis, severe sepsis, septic shock, inflammation, graft versus host disease, autoimmune diseases, acute respiratory distress syndrome, granulomatous diseases, chronic infections, transplant rejection, cachexia, bacterial infections, viral infections, parasitic infections, fungal infections, and/or trauma. It also plays a role in diseases mediated by microbial toxins including Gram-negative bacterial endotoxin, Gram-positive bacterial endotoxins, toxins of other microbial or infectious agents, mycobacteria (such as M. tuberculosis), and cell wall fragments of microbial pathogens such as peptidoglycan and lipoteichoic acids. MIF acts in concert with glucocorticoids to regulate inflammation and immunity, and plays a role in the development of a primary immune response. In particular, MIF potentiates lethality of endotoxemia, whereas the inhibition of MIF confers protection against lethal endotoxemia. Inhibition of MIF similarly confers protection against toxic shock syndrome. Surprisingly, MIF is induced by many of the glucocorticoids that are considered to be anti-inflammatory agents.

The resting pituitary contains large stores of preformed MIF which are released into the circulation after activation of the hypothalamic-pituitary axis by endotoxin, stress, and several cytokines and mediators, which participate in the proinflammatory response, such as TNF-α, IL-1β, IL-6 or IFN γ, to cite only a few. MIF is also released by macrophages in response to low doses of endotoxin; in response to TNF-α and IFN γ; in response to parasitic infection; in response to bacteria, mycobacteria, and toxins (see Table 2); and in response to steroids. Thus, the macrophage is not only a target for MIF, but is also an important source of MIF in vivo.

The inhibition of MIF activity and/or release may be used to treat inflammatory response and shock. Beneficial effects may be achieved by intervention at both early and late stages of the shock response. In this respect, the working examples also describe the production of monoclonal and polyclonal antibodies directed against both human and murine MIF, which may be used to neutralize MIF activity.

MIF also plays an important role during the post-acute stage of the shock response, and therefore, modulation of MIF activity provides an opportunity to intervene at late stages of shock where other treatments, such as anti-TNF-α therapy, are ineffective. MIF is released at the early and late stage of shock, when it is detectable in the circulation. In the experimental system used herein, anti-MIF therapy protected against lethal shock in animals challenged with high does of endotoxin, in animals challenged with TNF-α, and in animals challenged with bacteria. See Calandra et al., Nat. Med. 6:164-70 (2000). The ability of anti-MIF antibody therapy to protect animals challenged with TNFα: indicates that neutralization of MIF during the later, post-acute phase of septic shock is efficacious. The protective effect of the antibody may be attributed, in part, to neutralization of pituitary and macrophage MIF released in the post-acute phase of septic shock when circulating MIF is readily detected. Because MIF is a necessary component of shock syndrome, and because the peak of serum MIF expression occurs after that of TNF-α, MIF inhibitors may provide a successful treatment for mediator-induced diseases or pathology, useful after the time point at which administration of TNF-α inhibitors is no longer effective.

Because steroids are potent inhibitors of cytokine production, the effect of steroids on MIF secretion by macrophages and pituitary cells was examined. Surprisingly, steroids were found to induce rather than inhibit MIF secretion by these cells. Thus, the secretion of MIF in response to steroids may reduce the benefit of steroid therapy currently used to treat inflammation. Therefore, MIF inhibition therapy may be used in conjunction with steroids to treat shock and other mediator-induced pathological states, particularly in chronic inflammatory states such as rheumatoid arthritis. Such combination therapy may be beneficial even subsequent to the onset of pathogenic or other inflammatory responses. For example, administration of steroids alone inhibits the TNF-α. response only if given simultaneously with, or before, LPS challenge. In the clinical setting, the administration of steroids subsequent to the onset of septic shock symptoms has proven to be of little benefit. See Bone et al., 1987, N. Engl. J. Med. 317: 653-658; Spring et al., 1984; N. Engl. J. Med. 311: 1137-1141. Combination steroids/MIF inhibition therapy may be used to overcome this obstacle. When treating conditions such as rheumatoid arthritis, the administration of steroids alone can result in the induction of MIF, which may override the inhibitory effects of the steroid on the inflammatory response. In such cases, MIF inhibition therapy can be used in conjunction with steroid treatment. Therapy can be designed to inhibit MIF release and/or activity locally and/or systemically.

Because resting, nonstimulated pituitary cells contain significant amounts of preformed MIF, a large fraction of the MIF that is released by these cells in response to LPS arises from stored, intracellular pools. Electron microscopic studies of whole mouse pituitaries labeled with immunogold-conjugated anti-MIF antibody localize MIF to granules present within corticotrophic cells. It appears that in vivo, MIF-containing granules are released either by the direct action of circulating endotoxin or by specific hypothalamic releasing factor(s).

Whole tissue analyses demonstrated significant amounts of both MIF protein and mRNA in organs that have a high content of macrophages. This data complement observations that MIF mRNA is present constitutively in tissues such as the spleen, the liver, and the kidney. See Lanahan et al., 1992, Mol. Cell. Biol. 12: 3919-3929.

Various proinflammatory stimuli, such as LPS, TNF-α and IFN γ were observed to be potent inducers of macrophage MIF release. For example, secretion of significant amounts of MIF occurred at LPS concentrations (10-100 pg ml⁻¹) that are generally lower than has been observed to be optimal for the induction of TNF-α release. Significant amounts of preformed MIF mRNA were also present in resting macrophages, and stimulation by LPS increased these mRNA levels approximately two-fold. Peak mRNA stimulation also occurred at very low concentrations of LPS (1 pg ml⁻¹).

As noted, DNA homology analysis shows that both human and murine MIF lack a conventional N-terminal leader sequence. Thus, MIF joins a growing list of cytokines, such as IL-1 (Rubartelli et al., 1990, EMBO J. 9: 1503-1510), basic fibroblast growth factor (bFGF; Jackson et al., 1992, Proc. Natl. Acad. Sci. USA 89: 10691-10695), and a secreted form of cyclophilin (Sherry et al., 1992, Proc. Natl. Acad. Sci. USA 89: 3511-3515), which are released from cells by non-classical protein secretion pathways.

Inhibitors of MIF Activity

Factors that bind MIF and neutralize its biological activity (hereinafter referred to as MIF binding partners) may be used in accordance with the invention as treatments of conditions involving mediator-induced diseases or pathology. While levels of MIF protein may increase due to endotoxin challenge, the interaction of inhibitory MIF-binding partners with MIF protein prohibits a concomitant increase in MIF activity. Such factors include, but are not limited to anti-MIF antibodies, antibody fragments, MIF receptors, and MIF receptor fragments.

Various procedures known in the art may be used for the production of antibodies to epitopes of recombinantly produced (e.g., using recombinant DNA techniques described infra), or naturally purified MIF. Neutralizing antibodies, ie. those that compete for or sterically obstruct the binding sites of the MIF receptor, are especially preferred for diagnostics and therapeutics. Such antibodies include but are not limited to polyclonal, monoclonal, chimeric, single chain, Fab fragments, and fragments produced by an Fab expression library.

For the production of antibodies, various host animals may be immunized by injection with MIF and/or a portion of MIF. Such host animals may include but are not limited to rabbits, mice, and rats. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Monoclonal antibodies to MIF may be prepared by using any technique which provides for the production of antibody molecules by continuous cell lines in culture. These include but are not limited to the hybridoma technique originally described by Kohler and Milstein, (Nature, 1975, 256:495-497), the human B-cell hybridoma technique (Kosbor et al., 1983, Immunology Today, 4:72; Cote et al., 1983, Proc. Natl. Acad. Sci., 80:2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce MIF-specific single chain antibodies.

The hybridoma technique has been utilized to generate anti-MIF monoclonal antibodies. Hybridomas secreting IgG monoclonal antibodies directed against both human and murine forms of MIF have been isolated and characterized for their ability to neutralize MIF biological activity. Anti-MIF monoclonal antibodies were shown to inhibit the stimulation of macrophage-killing of intracellular parasites. The anti-MIF monoclonal antibodies have also been utilized to develop a specific and sensitive ELISA screening assay for MIF. Both the anti-MIF monoclonal antibodies and the ELISA assay may be used in the diagnosis and/or treatment of inflammatory responses and shock.

Antibody fragments that recognize specific MIF epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity to MIF.

MIF receptors, MIF receptor fragments, and/or MIF receptor analogs may, in accordance with the invention, be used as inhibitors of MIF biological activity. By binding to MIF protein, these classes of molecules may inhibit the binding of MIF to cellular MIF receptors, thus disrupting the mechanism by which MIF exerts its biological activity. Small organic molecules which mimic the activity of such molecules are also within the scope of the present invention.

MIF receptors may include any cell surface molecule that binds MIF in an amino acid sequence-specific and/or structurally-specific fashion. Such MIF receptors include, but are not limited to the 72 kD MIF receptor class having a partial amino acid sequence of:

AKKGAVGGI [SEQ ID NO: 1]

and the 52 kD receptor having a partial amino acid sequence of:

I-X-HNTVATEI(S)(G)YN(N/G)A(M) [SEQ ID NO: 2]. The residues in parenthesis represent provisional assignments.

Additional MIF receptors and genes that encode MIF receptors may be identified, isolated, and cloned using a variety of techniques well known to those of ordinary skill in the art. For example, MIF receptor molecules may be identified and isolated using standard affinity chromatography techniques wherein those molecules exhibiting sequence- and/or structural binding specificity to MIF protein are separated from other non-MIF binding molecules. The MIF binding proteins may be additionally purified, using standard techniques, at which point the protein may be tested and utilized for its ability to inhibit MIF.

Alternatively, the amino acid sequence of the purified protein may be at least partially determined, and then used to design oligonucleotides with which to screen cDNA and/or genomic libraries in order to clone the gene(s) that encode(s) MIF receptors, techniques which are well known to those of skill in the art. Further, new MIF receptor genes may be cloned by construction of a cDNA library in a mammalian expression vector such as pcDNA 1, that contains SV40 origin of replication sequences which permit high copy number expression of plasmids when transferred into COS cells. The expression of the MIF receptor on the surface of transfected COS cells may be detected in a number of ways, including the use of radioactive, fluorescent, or enzymatically labeled MIF. Cells expressing an MIF receptor may be enriched by subjecting transfected cells to a FACS (fluorescent activated cell sorter). For a review of cloning strategies which may be used, see e.g., Maniatis, 1989, Molecular Cloning, A Laboratory Manual, Cold Springs Harbor Press, N.Y.; and Ausubel et al., 1989, Current Protocols in Molecular Biology, (Green Publishing Associates and Wiley Interscience, N.Y.)

Fragments of any of the MIF receptors described above may also be used as MIF inhibitory agents, and any MIF receptor fragment possessing any amino, carboxy, and/or internal deletion that specifically binds MIF so as to inhibit MIF biological activity is intended to be within the scope of this invention. An amino and/or carboxy deletion refers to a molecule possessing amino and/or carboxy terminal truncations of at least one amino acid residue. An internal deletion refers to molecules that possess one or more non-terminal deletions of at least one amino acid residue. Among these MIF receptor fragments are truncated receptors in which the cytoplasmic or a portion of the cytoplasmic domain has been deleted, and fragments in which the cytoplasmic and the transmembrane domain(s) has been deleted to yield a soluble MIF receptor containing all or part of the MIF receptor extracellular domain.

MIF receptor analogs, which specifically bind MIF may also be used to inhibit MIF activity. Such MIF receptor analogs may include MIF receptor or receptor fragments further possessing one or more additional amino acids located at the amino terminus, carboxy terminus, or between any two adjacent MIF receptor amino acid residues. The additional amino acids may be part of a heterologous peptide functionally attached to all or a portion of the MIF receptor protein to form a MIF receptor fusion protein. For example, and not by way of limitation, the MIF receptor, or a truncated portion thereof, can be engineered as a fusion protein with a desired Fc portion of an immunoglobulin. MIF receptor analogs may also include MIF receptor or MIF receptor fragments further possessing one or more amino acid substitutions of a conservative or non-conservative nature. Conservative amino acid substitutions consist of replacing one or more amino acids with amino acids of similar charge, size, and/or hydrophobicity characteristics, such as, for example, a glutamic acid (E) to aspartic acid (D) amino acid substitution. Non-conservative substitutions consist of replacing one or more amino acids with amino acids possessing dissimilar charge, size, and/or hydrophobicity characteristics, such as, for example, a glutamic acid (E) to valine (V) substitution.

The MIF receptors, MIF receptor fragments and/or analogs may be made using recombinant DNA techniques. Here, the nucleotide sequences encoding the peptides of the invention may be synthesized, and/or cloned, and expressed according to techniques well known to those of ordinary skill in the art. See, for example, Ausubel, F. M. et al., eds., 1989, Current Protocols In Molecular Biology, Vol. 1 and 2, John Wiley and Sons, New York. Caruthers, et al., 1980, Nuc. Acids Res. Symp. Ser. 7:215-233; Crea and Horn, 1980, Nuc. Acids Res. 9(10):2331; Matteucci and Caruthers, 1980, Tetrahedron Letters 21:719; and Chow and Kempe, 1981, Nuc. Acids Res. 9(12):2807-2817.

Alternatively, the protein itself could be produced using chemical methods to synthesize the amino acid sequence in whole or in part. For example, peptides can be synthesized by solid phase techniques, cleaved from the resin, and purified by preparative high performance liquid chromatography. (e.g., see Creighton, 1983, Proteins Structures And Molecular Principles, W. H. Freeman and Co., N.Y. pp. 50-60.) The composition of the synthetic peptides may be confirmed by amino acid analysis or sequencing (e.g., the Edman degradation procedure; see Creighton, 1983, Proteins, Structures and Molecular Principles, W. H. Freeman and Co., N.Y., pp. 34-49).

These molecules may also be synthesized utilizing alternative procedures, which may advantageously affect certain of the molecules' properties, such as stability, bioavailability, and MIF inhibitory activity. For example, MIF receptors, MIF receptor fragments, and MIF receptor analogs may be synthesized such that one or more of the bonds, which link the amino acid residues of the peptides are non-peptide bonds. These alternative non-peptide bonds may be formed by utilizing reactions well known to those in the art, and may include, but are not limited to imino, ester, hydrazide, semicarbazide, and azo bonds, to name but a few. In another embodiment, the proteins may be synthesized with additional chemical groups present at their amino and/or carboxy termini. For example, hydrophobic groups such as carbobenzoxyl, dansyl, or t-butyloxycarbonyl groups, may be added to the peptides' amino terminus. Further, the peptides may be synthesized such that their steric configuration is altered. For example, the D-isomer of one or more of the amino acid residues of the peptide may be used, rather than the usual L-isomer.

MIF-receptor Antagonists

Molecules which inhibit MIF biological activity by binding to MIF receptors may also be utilized for the treatment of conditions involving a mediator-induced diseases or pathology. Such molecules may include, but are not limited to anti-MIF receptor antibodies and MIF analogs.

Anti-MIF receptor antibodies may be raised and used to neutralize MIF receptor function. Antibodies against all or any portion of a MIF receptor protein may be produced by any method known to those skilled in the art.

MIF analogs may include molecules that bind the MIF receptor but do not exhibit biological activity. Such analogs compete with MIF for binding to the MIF receptor, and, therefore, when used in vivo, may act to block the effects of MIF in the progress of a mediator-induced diseases or pathology. A variety of techniques well known to those of skill in the art may be used to design MIF analogs. Recombinant DNA techniques may be used to produce modified MIF proteins containing, for example, amino acid insertions, deletions and/or substitutions which yield MIF analogs with receptor binding capabilities, but no biological activity. Alternatively, MIF analogs may be synthesized using chemical methods known to those of ordinary skill in the art. See, for example, Sambrook et al., 1989, Molecular Cloning, A Laboratory Manual, Cold Spring Harbor Press, N.Y.

MIF receptors and/or cell lines that express MIF receptors may be used to identify and/or assay potential MIF antagonists. For example, one method that may be pursued in the identification of such MIF antagonist molecules involves attaching MIF receptor molecules to a solid matrix, such as agarose or plastic beads, microtiter wells, or petri dishes, using techniques well known to those of skill in the art, and subsequently incubating the attached MIF receptor molecules in the presence of a potential MIF analog or analogs. After incubating, unbound compounds are washed away, and the MIF receptor-bound compounds are recovered. In this procedure, large numbers of types of molecules may be simultaneously screened for MIF receptor-binding activity. Bound molecules may be eluted from the MIF receptor molecules by, for example, competing them away from the MIF receptor molecules with the addition of excess MIF, changing the pH or osmotic strength of the buffer or adding chaotropic agents. The binding/elution steps bring about a partial purification of the molecules of interest.

In order to continue the purification process, the eluted molecules may be further fractionated by one or more chromatographic and/or other separation techniques well known in the art until the molecule(s) of interest is (are) purified to the extent necessary. Once compounds having MIF-receptor binding capabilities are identified, the compounds may be assayed for their effects on mediator-induced diseases or pathology using cell lines such as those described above, or by normal experimental animal models or alternatively, by utilizing transgenic animal models.

Alternatively, screening of peptide libraries with recombinantly produced MIF receptors and/or MIF receptor fragments may be used to identify potential MIF analogs. Once peptides that bind MIF receptor are identified using this screening technique, their effects on mediator-induced diseases or pathology may be assayed using cells lines such as those described herein, or alternatively, may be evaluated using normal experimental animal models or transgenic animals. Small organic molecules, which mimic and/or inhibit the activity of such peptides are also within the scope of the present invention.

Random peptide libraries consist of all possible combinations of amino acids, and such libraries may be attached to a suitable small particulate solid phase support and used to identify peptides that are able to bind to a given receptor. See Lam, K. S. et al., 1991, Nature 354: 82-84. The screening of peptide libraries may have therapeutic value in the discovery of pharmaceutical agents that act to inhibit the biological activity of receptors through their interactions with the given receptor.

Identification of molecules that are able to bind to the MIF receptor may be accomplished by screening a peptide library with recombinant soluble MIF receptor protein. Methods for expression and purification of molecules such as MIF receptors are well known to those of skill in the art. For screening, it is preferred to label or “tag” the MIF receptor molecule. The protein may be conjugated to enzymes such as alkaline phosphatase or horseradish peroxidase or to other reagents such as fluorescent labels, which may include fluorescein isothyiocynate (FITC), phycoerythrin (PE) or rhodamine. Conjugation of any given label to the MIF receptor may be performed using techniques that are routine in the art. Alternatively, MIF receptor expression vectors may be engineered to express a chimeric MIF receptor protein containing an epitope for which a commercially available antibody exists. The epitope-specific antibody may be tagged using methods well known in the art including labeling with enzymes, fluorescent dyes or colored or magnetic beads.

The “tagged” MIF receptor or receptor/conjugate is incubated with the random peptide library for 30 minutes to one hour at 22° C. to allow complex formation between MIF receptor and peptide species within the library. The library is then washed to remove any unbound MIF receptor protein. If the MIF receptor has been conjugated to alkaline phosphatase or horseradish peroxidase the whole library is poured into a petri dish containing a substrates for either alkaline phosphatase or peroxidase, for example, 5-bromo-4-chloro-3-indoyl phosphate (BCIP) or 3,3′,4,4″-diaminobenzidine (DAB), respectively. After incubating for several minutes, the peptide/solid phase-MIF receptor complex changes color, and can be easily identified and isolated physically under a dissecting microscope with a micromanipulator. If a fluorescent tagged MIF receptor molecule has been used, complexes may be isolated by fluorescence-activated sorting. If a chimeric MIF protein expressing a heterologous epitope has been used, detection of the peptide/MIF receptor complex may be accomplished by using a labeled epitope-specific antibody. Once isolated, the MIF receptor conjugate may be eluted off, the peptide support washed, and the identity of the peptide attached to the solid phase support determined by peptide sequencing.

MIF analogs may also be identified using cell lines that express MIF receptor. Such cell lines may be ones, which naturally express the receptor, such as RAW 264.7 cells, or alternatively, cell lines that have been engineered using recombinant techniques to express MIF receptor. These cell lines may also be used to evaluate potential MIF analogs identified using MIF receptor binding techniques such as those described above.

With respect to engineered cell lines, a variety of cells may be utilized as host cells for expression of the recombinant MIF receptor, including, but not limited to animal cell systems infected with recombinant virus expression vectors (e.g., adeno-virus, vaccinia virus) including cell lines engineered to contain multiple copies of the MIF receptor DNA either stably amplified (e.g., CHO/dhfr) or unstably amplified in double-minute chromosomes (e.g., murine cell lines).

In cases where an adenovirus is used as an expression vector, the MIF receptor-coding sequence may be ligated to an adenovirus transcription-translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing MIF receptor in infected hosts (e.g., See Logan & Shenk, 1984, Proc. Natl. Acad. Sci. (USA) 81:3655-3659). Alternatively, the vaccinia 7.5K promoter may be used. See, e.g., Mackett et al., 1982, Proc. Natl. Acad. Sci. (USA) 79: 7415-7419; Mackett et al., 1984, J. Virol. 49: 857-864; Panicali et al., 1982, Proc. Natl. Acad. Sci. 79: 4927-4931.

Specific initiation signals may also be required for efficient translation of inserted MIF receptor coding sequences. These signals include the ATG initiation codon and adjacent sequences. In cases where the entire MIF receptor gene, including its own initiation codon and adjacent sequences, is inserted into the appropriate expression vector, no additional transcriptional control signals may be needed. However, in cases where only a portion of the MIF receptor coding sequence is inserted, exogenous transcriptional control signals, including the ATG initiation codon, must be provided. Furthermore, the initiation codon must be in phase with the reading frame of the MIF receptor coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. See Bittner et al., 1987, Methods in Enzymol. 153: 516-544.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells, which possess the cellular machinery for proper processing of the primary transcript, and for any normal glycosylation, and/or phosphorylation of the gene product may be used. Such mammalian host cells include but are not limited to CHO, VERO, BHK, HeLa, COS, MDCK, 293, W138, etc.

The cell lines may be utilized to screen and identify MIF analogs. Synthetic compounds, natural products, and other sources of potentially biologically active materials can be screened in a number of ways, by, for example, testing a compound's ability to inhibit binding of MIF to a MIF receptor. Standard receptor binding techniques may be utilized for this purpose.

The ability of anti-MIF receptor antibodies and potential MIF analogs to reduce or inhibit MIF biological activity may be assayed in vivo by utilizing animals expressing MIF receptor, for instance, normal animals. Such animals may also include transgenic animal models such as those described below.

Other Inhibitors of MIF Activity

As explained above, certain steroids, commonly thought to be either inactive or “anti-steroidal” actually inhibit the release of MIF, e.g., 20α dihydrocortisol. These steroids, or any other compound which inhibits the release of preformed MIF, can be used in combination therapy with anti-inflammatory steroids.

Compounds which inhibit the release of MIF can be identified in cell based assays. In general, any pituitary or macrophage cell line that releases MIF in response to a challenge dose of steroid can be used. The assay can be conducted by adding the test compound to the cells in culture, which are then challenged with a dose of steroid known to induce MIF release. Test compounds may be administered simultaneously with, or up to several hours before or after the challenge dose so as to identify agents that are useful in inhibiting the MIF response at different stages, i.e., inhibiting release of pre-formed MIF, versus inhibiting de novo synthesis and release, versus inhibiting both.

The conditioned media is then collected from the cultured cells and assayed for MIF; e.g., by immunoassay, including but not limited to an ELISA, Western blot, radioimmunoassay, etc. A reduced amount of MIF in the conditioned media indicates that the test compound inhibits the steroid-induced release of MIF. Compounds so identified in this assay may be used in combination therapy with steroids to treat inflammation. “Biologically inert” or innocuous compounds, such as the inactive steroids, or steroids which can be used at doses that do not cause undesired side effects, may be preferred for therapeutic use. However, any inhibitory compounds having a good therapeutic index, e.g., low toxicity and little or no side effects may be used.

Dose and Treatment Regimens

Inhibitors of MIF biological activity such as anti-MIF antibodies, MIF receptors, MIF receptor fragments, MIF receptor analogs, anti-MIF receptor antibodies, MIF analogs and inhibitors of MIF release, may be administered using techniques well known to those in the art. Preferably, agents are formulated and administered systemically. Techniques for formulation and administration may be found in “Remington's Pharmaceutical Sciences”, 18th ed., 1990, Mack Publishing Co., Easton, Pa. Suitable routes may include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few. Most preferably, administration is intravenous. For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Effective concentrations and frequencies of dosages of the MIF inhibitory compounds invention to be administered may be determined through procedures well known to those in the art, which address such parameters as biological half-life, bioavailability, and toxicity. In the case of anti-MIF antibodies, a preferred dosage concentration may range from about 0.1 mg/kg body weight to about 20 mg/kg body weight, with about 10 mg/kg body weight being most preferred. Because antibodies typically exhibit long half-lives in circulation, a single administration of antiserum may be sufficient to maintain the required circulating concentration. In the case of compounds exhibiting shorter half-lives, multiple doses may be necessary to establish and maintain the requisite concentration in circulation.

MIF inhibitors may be administered to patients alone or in combination with other therapies. Such therapies include the sequential or concurrent administration of inhibitors or antagonists of initiators of mediator-induced diseases or pathology (e.g anti-LPS), inhibitors or antagonists of participants in the endogenous cytokine responses (e.g. anti-TNF-α, anti-IL-1, anti-IFN γ, IL-1 RA); and compounds that inhibit or antagonize mediator-induced diseases or pathology directly (e.g. steroids, glucocorticoids or IL-10). MIF inhibitor dosage concentration and frequency may be altered when used as a part of a combination therapy and, therefore appropriate tests must be performed in order to determine the best dosage when more than one class of inhibitory compounds is to be administered.

Because MIF expression reaches a peak level in response to endotoxin challenge later than TNF-α, anti-MIF compounds may be administered after the period within which anti-TNF-α inhibitors are effective. Anti-MIF antibody conferred full protection against endotoxemia in animals. Anti-MIF antibody has also been shown to reduce circulating TNF-α levels, indicating that anti-MIF antibody inhibits the proinflammatory spectrum of activity of MIF, and that anti-MIF antibody inhibits the inflammatory cytokine cascade generally. In in vivo experiments, moreover, anti-MIF antibody was shown to protect against lethal shock induced by administration of exogenous TNF-α. Therefore, the beneficial effect of the anti-MIF antibody probably resides in its ability to neutralize the bioactivity of both macrophage MIF released during the acute phase in response to a proinflammatory stimulus (e.g., LPS, or TNF-α and IFN γ), and MIF released by both the pituitary and macrophages during the post-acute phase of the shock response.

The development of anti-MIF monoclonal antibodies provides a specific means for disrupting the mechanism by which MIF exerts its biological activity. The anti-MIF monoclonal antibodies may be used as a therapeutic for conditions involving MIF-mediated adverse effects generally, for instance endotoxin lethality and mediator-induced lethality, including TNF-αtoxicity, such as observed during septic shock. The same antibodies may also be used to protect against the toxic effects of nitric oxide production by macrophages, which is also induced by MIF.

MIF has been shown to be an important mediator in the immune response to malaria infection, therefore anti-MIF monoclonal antibodies may be effective in ameliorating the lethality of parasite-induced cytokine release. Aside from being an important mediator of the inflammatory immune response, MIF has also been shown to be involved in the development of a primary immune response. Furthermore, the administration of anti-MIF monoclonal antibodies has been shown to abrogate an antigen-specific immune response, confirming that anti-MIF antibodies may be useful therapeutic agents for substantially reducing an undesired immune reaction, such as allergy or autoimmunity.

Inhibitors of MIF and/or MIF Receptor Gene Expression

Nucleotide sequences derived from the coding, non-coding, and/or regulatory sequences of the MIF and/or MIF receptor genes may be used to prevent or reduce the expression of these genes, leading to a reduction or inhibition of MIF activity. The nucleotide sequence encoding the human MIF protein has been reported. In addition, the nucleotide sequence of human MIF has been corrected (See U.S. Pat. No. 6,080,407), and a cDNA corresponding to the nucleotide sequence encoding the murine MIF protein has now been identified. Further, the MIF receptor amino acid sequence may, for example, be used to design oligonucleotides for the regulation of MIF receptor genes. Among the techniques by which such regulation of gene expression may be accomplished are, as described below, antisense, triple helix, and ribozyme approaches. Administration of these nucleotide sequences, therefore, may be used to temporarily block expression and/or transcription of the MIF and/or MIF receptor genes as one method of treatment for conditions involving mediator-induced toxicity.

These approaches which target gene expression may be used alone, in combination with each other, or alternatively, in conjunction with any of the inhibitory MIF-binding and/or MIF receptor antagonist procedures described above. Further, these gene regulation approaches may be used together with anti-TNF-α, anti-initiators and/or other anti-cytokine therapies.

Anti-sense RNA and Ribozymes

Within the scope of the invention are oligoribonucleotide sequences, that include anti-sense RNA and DNA molecules and ribozymes that function to inhibit the translation of MIF and/or MIF receptor mRNA. Anti-sense RNA and DNA molecules act to directly block the translation of mRNA by binding to targeted mRNA and preventing protein translation, either by inhibition of ribosome binding and/or translocation or by bringing about the nuclease degradation of the mRNA molecule itself.

Various modifications to the DNA molecules may be introduced as a means of increasing intracellular stability and half-life. Possible modifications include but are not limited to the addition of flanking sequences of ribo- or deoxy- nucleotides to the 5′ and/or 3′ ends of the molecule or the use of phosphorothioate or 2′ O-methyl rather than phosphodiesterase linkages within the oligodeoxyribonucleotide backbone.

An “antisense” nucleic acid comprises a nucleotide sequence that is complementary to a “sense” nucleic acid encoding a protein (e.g., complementary to the coding strand of a double-stranded cDNA molecule or complementary to an mRNA sequence). Antisense nucleic acids can be designed according to the rules of Watson and Crick or Hoogsteen base pairing. The antisense nucleic acid molecule can be complementary to the entire coding region of an mRNA, but more preferably is an oligonucleotide that is antisense to only a portion of the coding or noncoding region of an mRNA. An antisense oligonucleotide can be, for example, about 5, 10, 15, 20, 25, 30, 35, 40, 45 or 50 nucleotides in length. An antisense nucleic acid can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Antisense nucleic acid molecules are typically administered to a subject or generated in situ such that they hybridize with or bind to cellular mRNA and/or genomic DNA encoding a protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

The antisense nucleic acid molecule may be an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

The antisense nucleic acid may be a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave mRNA transcripts to thereby inhibit translation of the mRNA. A ribozyme having specificity for a nucleic acid can be designed based upon the nucleotide sequence of a cDNA. For example, a derivative of a Tetrahymena L-19 IVS RNA can be constructed in which the nucleotide sequence of the active site is complementary to the nucleotide sequence to be cleaved in an mRNA. See, e.g., U.S. Pat. No. 4,987,071 to Cech, et al. and U.S. Pat. No. 5,116,742 to Cech, et al. mRNA can also be used to select a catalytic RNA having a specific ribonuclease activity from a pool of RNA molecules. See, e.g., Bartel et al., (1993) Science 261:1411-1418.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Within the scope of the invention are engineered hammerhead motif ribozyme molecules that specifically and efficiently catalyze endonucleolytic cleavage of MIF and/or MIF receptor mRNA sequences.

Specific ribozyme cleavage sites within any potential RNA target are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complimentary oligonucleotides, using ribonuclease protection assays.

Both anti-sense RNA and DNA molecules and ribozymes of the invention may be prepared by any method known in the art for the synthesis of nucleic acid molecules. These include techniques for chemically synthesizing oligodeoxyribonucleotides well known in the art such as, for example, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Triplex DNA Formation

Oligodeoxyribonucleotides can form sequence-specific triple helices by hydrogen bonding to specific complementary sequences in duplexed DNA. Interest in triple helices has focused on the potential biological and therapeutic applications of these structures. Formation of specific triple helices may selectively inhibit the replication and/or gene expression of targeted genes by prohibiting the specific binding of functional trans-acting factors.

Oligonucleotides to be used in triplex helix formation should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Oligonucleotide sequences may be pyrimidine-based, which will result in TAT and CGC triplets across the three associated strands of the resulting triple helix. The pyrimidine-rich oligonucleotides provide base complementarity to a purine-rich region of a single strand of the duplex in a parallel orientation to that strand. In addition, oligonucleotides may be chosen that are purine-rich, for example, containing a stretch of G residues. These oligonucleotides will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation may be increased by creating a so-called “switchback” oligonucleotide. Switchback oligonucleotides are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Alternatively, gene expression can be inhibited by targeting nucleotide sequences complementary to the regulatory region of a nucleic acid (e.g., the promoter and/or enhancers) to form triple helical structures that prevent transcription of the gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al, 1996. supra; Perry-O'Keefe, et al., 1996. Proc. Natl. Acad. Sci. USA 93: 14670-14675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO89/10134). In addition, oligonucleotides can be modified with hybridization triggered cleavage agents (see, e.g., Krol, et al., 1988. Bio Techniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

Administration of Oligonucleotides

For anti-MIF therapeutic uses, the inhibitory oligonucleotides may be formulated and administered through a variety of means, including systemic, and localized, or topical, administration. Techniques for formulation and administration may be found in “Remington's Pharmaceutical Sciences,” Mack Publishing Co., Easton, Pa., latest edition. The mode of administration may be selected to maximize delivery to a desired target organ in the body. For example, ¹²⁵I-MIF binding studies indicate that MIF is preferentially distributed to the liver and kidney. Therefore, oligonucleotides designed to inhibit expression of the MIF-receptor may be formulated for targeting to these organs; in this regard, liposome-encapsulated oligonucleotides may prove beneficial. Alternatively, MIF itself is produced in T cells and macrophages and, in response to endotoxin induction, in the pituitary. Therefore, oligonucleotides designed to inhibit the expression of MIF should be formulated for targeting to these organs.

For systemic administration, injection is preferred, including intramuscular, intravenous, intraperitoneal, and subcutaneous. For injection, the oligonucleotides of interest are formulated in aqueous solutions, preferably in physiologically compatible buffers such as Hanks's solution, Ringer's solution, or physiological saline buffer. In addition, the oligonucleotides may be formulated in solid or lyophilized form, then redissolved or suspended immediately prior to use. Systemic administration may also be accomplished by transmucosal, transdermal, or oral means. For transmucosal or transdermal administration, penetrants appropriate to the barrier to be permeated are used in the formulation. Such penetrants are generally known in the art. Transmucosal administration may be through nasal sprays or suppositories. For oral administration, oligonucleotides may be formulated into capsules, tablets, and tonics. For topical administration, the oligonucleotides of the invention are formulated into ointments, salves, gels, or creams, as is generally known to those of ordinary skill in the art.

Alternatively, the oligonucleotides of the invention may first be encapsulated into liposomes, then administered as described above. Liposomes are spherical lipid bilayers with aqueous interiors. All molecules that are present in an aqueous solution at the time of liposome formation (in this case, oligonucleotides) are incorporated into this aqueous interior. The liposomal contents are both protected from the external microenvironment and, because liposomes fuse with cell membranes, are efficiently delivered into the cell cytoplasm, obviating the need to neutralize the oligonucleotides' negative charge.

The introduction of oligonucleotides into organisms and cells for such purposes may be accomplished by several means. For mammalian administration, each of the techniques described above for therapeutic oligonucleotide purposes may be used. In addition, other standard techniques for introduction of nucleic acids into cells, including, but not limited to, electroporation, microinjection, and calcium phosphate precipitation techniques may be utilized.

Transgenic Animal Models

As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Transgenic animals may be engineered, using techniques well known to those of skill in the art, whose cells may contain modified and/or additional MIF and or MIF receptor genes within their genomes. For example, animals may be produced that contain inactive MIF and/or MIF receptor genes, or alternatively, may contain additional MIF and/or MIF receptor genes.

Such transgenic animals may be used as model systems for the evaluation of cytokine responses in vivo, and may additionally serve as a means by which new drugs for the treatment of conditions involving mediator-induced toxicity are identified and tested.

DNA containing the nucleotide coding sequence for all or any portion of the MIF gene may be used to produce transgenic animals. Alternatively, all or any portion of a gene encoding a MIF receptor may be used. Further, insertions, substitutions, and/or deletions of one or more nucleotides of the MIF and/or MIF receptor genes may also be utilized in the construction of the transgenic animals. Due to the degeneracy of the genetic code, other DNA sequences which encode substantially the same MIF or MIF receptor protein or a functional equivalent can also be used. The nucleotide coding sequence used to produce the transgenic animals of the invention may be regulated by any known promoter regulatory nucleotide sequence. If it is required that expression of the transgene be limited to one or more specific tissues, tissue-specific enhancer regulatory sequences may also be used. Multiple copies of the genes or gene constructs may be stably integrated into the transgenic founder animals.

In order to produce the gene constructs used in the invention, recombinant DNA and cloning methods, which are well known to those skilled in the art may be utilized (see Sambrook et al., 1989, Molecular Cloning, A Laboratory Manual, 2nd Ed., Cold Spring Harbor Laboratory Press, NY). In this regard, appropriate MIF and/or MIF receptor coding sequences may be generated from MIF cDNA or genomic clones using restriction enzyme sites that are conveniently located at the relevant positions within the sequences. Alternatively, or in conjunction with the method above, site directed mutagenesis techniques involving, for example, either the use of vectors such as M13 or phagemids, which are capable of producing single stranded circular DNA molecules, in conjunction with synthetic oligonucleotides and specific strains of Escherichia coli (E. coli) (Kunkel, T. A. et al., 1987, Meth. Enzymol. 154:367-382) or the use of synthetic oligonucleotides and PCR (polymerase chain reaction) (Ho et al., 1989, Gene 77:51-59; Kamman, M. et al., 1989, Nucl. Acids Res. 17: 5404) may be utilized to generate the necessary nucleotide coding sequences. Appropriate MIF and/or MIF receptor sequences may then be isolated, cloned, and used directly to produce transgenic animals. The sequences may also be used to engineer the chimeric gene constructs that utilize regulatory sequences other than the MIF and/or MIF receptor promoter, again using the techniques described here. These chimeric gene constructs would then also be used in the production of transgenic animals.

Transgenic animals may also be produced in which the function of the endogenous MIF and/or MIF receptor genes has been disrupted. To accomplish these endogenous gene disruptions, the technique of site-directed inactivation via gene targeting (Thomas, K. R. and Capecchi, M. R., 1987, Cell 51:503-512) may be used. Briefly, vectors containing some nucleotide sequences homologous to the endogenous gene of interest are designed for the purpose of integrating, via homologous recombination with chromosomal sequences, into and disrupting the function of, the nucleotide sequence of said endogenous gene.

Animals of any species, including but not limited to mice, rats, rabbits, guinea pigs, pigs, mini-pigs, and non-human primates, e.g., baboons, squirrel monkeys and chimpanzees may be used to generate the transgenic animals of the invention. Any technique known in the art may be used to introduce the transgene into animals to produce the founder lines of transgenic animals. Such techniques include, but are not limited to pronuclear microinjection (Hoppe, P. C. and Wagner, T. E., 1989, U.S. Pat. No. 4,873,191); retrovirus-mediated gene transfer into germ lines (Van der Putten et al., 1985, Proc. Natl. Acad. Sci., USA 82:6148-6152); gene targeting in embryonic stem cells (Thompson et al., 1989, Cell 56:313-321); electroporation of embryos (Lo, 1983, Mol Cell. Biol. 3:1803-1814); and sperm-mediated gene transfer (Lavitrano et al., 1989, Cell 57:717-723); etc. (For a review of such techniques, see Gordon, 1989, Transgenic Animals, Intl. Rev. Cytol. I 15:171-229, which is incorporated by reference herein in its entirety.)

Once the founder animals are produced, they may be bred, inbred, outbred, or crossbred to produce colonies of the particular genotype desired. Examples of such breeding strategies include but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic mice to produce mice homozygous for a given integration site in order to both augment expression and eliminate the need for screening of animals by DNA analysis; crossing of separate homozygous lines to produce compound heterozygous or homozygous lines; breeding animals to different inbred genetic backgrounds so as to examine effects of modifying alleles on expression of the transgene.

The present invention provides for transgenic animals that carry the transgene in all their cells, as well as animals which carry the transgene in some, but not all their cells, i.e., mosaic animals. The transgene may be integrated as a single transgene or in concatamers, e.g., head-to-head tandems or head-to-tail tandems.

A transgenic animal of the invention can be created by introducing a nucleic acid into the male pronuclei of a fertilized oocyte (e.g., by microinjection, retroviral infection) and allowing the oocyte to develop in a pseudopregnant female foster animal. Alternatively, a non-human homologue of the human gene, such as a mouse gene, can be isolated based on hybridization to the human cDNA and used as a transgene. Intronic sequences and polyadenylation signals can also be included in the transgene to increase the efficiency of expression of the transgene. A tissue-specific regulatory sequence(s) can be operably-linked to the transgene to direct expression of the protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the transgene in its genome and/or expression of mRNA in tissues or cells of the animals. A transgenic founder animal can then be used to breed additional animals carrying the transgene. Moreover, transgenic animals carrying a transgene-encoding protein can further be bred to other transgenic animals carrying other transgenes.

To create a homologous recombinant animal, a vector is prepared which contains at least a portion of a gene into which a deletion, addition or substitution has been introduced to thereby alter, e.g., functionally disrupt, the gene. The gene can be a human gene, but more preferably, is a non-human homologue of a human gene. The vector may be designed such that, upon homologous recombination, the endogenous gene is functionally disrupted (i.e., no longer encodes a functional protein; also referred to as a “knock out” vector).

Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′- and 3′-termini by additional nucleic acid of the gene to allow for homologous recombination to occur between the exogenous gene carried by the vector and an endogenous gene in an embryonic stem cell. The additional flanking nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is then introduced into an embryonic stem cell line (e.g., by electroporation) and cells in which the introduced gene has homologously-recombined with the endogenous gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxp recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Diagnostic Applications

MIF protein and/or mRNA levels may be monitored in an individual, using standard techniques, as an indication of the deleterious aspects of a disease condition. For the measurement of MIF protein concentrations, such monitoring techniques include, but are not limited to immunological assays such as, for example, Western blots, immunoassays such as radioimmuno-precipitation, enzyme-linked immunoassays, and the like. For the measurement of MIF mRNA concentrations, such techniques may include, for example, hybridization techniques such as Northern blot analysis, or any RNA amplification techniques, which may involve, for example, polymerase chain reaction (the experimental embodiment set forth in Mullis, K. B., 1987, U.S. Pat. No. 4,683,202) ligase chain reaction (Barany, F., 1991, Proc. Natl. Acad. Sci. USA 88: 189-193) self-sustained sequence replication (Guatelli, J. C. et al., 1990, Proc. Natl. Acad. Sci. USA 87: 1874-1878), transcriptional amplification system (Kwoh, D. Y. et al., 1989, Proc. Natl. Acad. Sci. USA 86: 173-1177), or Q-Beta Replicase (Lizardi, P. M. et al., 1988, Bio/Technology 6: 1197).

Antibodies

The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region that is located on the surface of the protein, e.g., a hydrophilic region. A hydrophobicity analysis of a protein sequence will indicate which regions of a polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

A protein, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen, which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem. 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

Antibodies directed against the protein antigens can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues, which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals, which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

Bispecific Antibodies

Bispecific antibodies are monoclonal, preferably human or humanized, antibodies that have binding specificities for at least two different antigens. One of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J. 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers, which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers. Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIll (CD1 6) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Pharmaceutical Compositions of Antibodies

Antibodies specifically binding a protein, as well as other molecules identified by the screening assays, can be administered for the treatment of various disorders in the form of pharmaceutical compositions. Principles and considerations involved in preparing such compositions, as well as guidance in the choice of components are provided, for example, in Remington : The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT ™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

Pharmaceutical Compositions

Pharmaceutical compositions suitable for administration typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

The invention will be further described in the following examples, which do not limit the scope of the invention described in the claims.

### EXAMPLE 1

### Reagents and Bacteria

Escherichia coli O 111:B4 LPS, FITC-labelled Serratia marcescens LPS, phorbol 12-myristate 13-acetate (PMA), and calcium ionophore A23187 were obtained from Sigma. G418 was from Brunschwig (Basel, Switzerland) and S. auereus peptidoglycan was from Fluka. Zymosan was from Molecular Probes. Peptidoglycan and zymosan contained less than 10 pg of endotoxin per microgram of material. E. coli O111 and O18, P. aeruginosa, K pneumoniae, S. aureus and group A streptococci (S. pyogenes) were grown overnight, enumerated, heat killed, divided into aloquots, and frozen at −20° C.

### EXAMPLE 2

### Cells, Constructs and Transfections

Mouse RAW 264.7 macrophages (American Type Culture Collection) were cultured in RPMI 1640 medium containing 2 mM glutamine (Gibco BRL), 10% heat-inactivated fetal calf serum (Seromed, Berlin), 100 IU/ml penicillin and 100 μg/ml streptomycin (Gibco BRL). Resident and thioglycolate-elicited peritoneal macrophages were obtained from MIF knockout. (See Bozza et al., J. Exp. Med. 189:341-46 (1999)) and MIF wild-type mice as described. (See Calandra et al, J. Exp. Med. 179:1895-1902 (1994)). Murine MIF cDNA was cloned in a 3′-5′ orientation in the cytomegalovirus promoter-containing expression vector pBK (Stratagene). See Waeber et al., Proc. Natl. Acad. Sci. USA 94 (1997). RAW 264.7 macrophages were transfected with the parental pBK vector or the recombinant antisense MIF vector using DOTAP (Roche Diagnostics). Stable transfected cell lines were selected using 250 μg/ml of G418 (Gibco BRL) and clones obtained by limited dilution. The MIF content of stable antisense MIF macrophages was analyzed by Western blotting and ELISA as described. See Calandra et al., J. Exp. Med. 179:1895-1902 (1994); Calandra et al., Proc. Natl. Acad. Sci. USA 95:11383-11388 (1998). For add-back experiments, antisense MIF RAW 264.7 macrophages were transiently transfected with 2 μg of mouse MIF expression construct obtained by cloning the coding sequence of mouse MIF into the pBK vector (Stratagene). The coding regions of mouse TLR2 and TLR4 (amino acids 20-784 for TLR2 and 25-835 for TLR4) were amplified by PCR from RAW 264.7 macrophages, cloned into the pGEM-T easy vector (Promega), sequenced and subcloned in the pFlag-CMV expression plasmid (Sigma). Proper expression of the constructs was verified by Western blot using anti-Flag antibodies. A truncated form of mouse TLR4 (amino acids 25-770) with a deletion of the C-terminal 65 amino acids of the intracytoplasmic domain of the molecule was obtained by digesting TLR4 DNA insert at the EcoNI site and at the BamHI site present in the 3′ polylinker of pFlag-CMV.

### EXAMPLE 3

### RNA Analysis

Total RNA was extracted from control and antisense MIF RAW 264. 7 macrophages and from MIF^{+/+} and MIF^{-/-} peritoneal macrophages. Expression of TNF-α, CD14, TLR2, TLR4, β-actin and GAPDH mRNA was assessed by Northern blotting using specific cDNA probes. Gene-specific mRNA signals were quantified using an Instant Imager 2024 (Packard).

### EXAMPLE 4

### Cytokine Measurement

The concentrations of TNF-α and IL-6 were determined using WEH1164 clone 13 mouse fibrosarcoma cells (TNF-α) and 7TD1 IL-6-dependent mouse-mouse hybridoma cells (IL-6). See Calandra et al., Nat. Med. 6:164-70 (2000).

### EXAMPLE 5

### Electromobility Shift Assay (EMSA)

Nuclear extracts of control or LPS-stimulated RAW 264.7 or antisense MIF macrophages were analyzed by EMSA using a consensus NF-κB probe (Santa Cruz) and a PU.1 probe derived from the sequence of the distal PU.1 binding site of the mouse Tlr4 promoter.

### EXAMPLE 6

### Mouse TLR4 Promoter

A 3-kb fragment corresponding to region −2715/+223 from the mouse Tlr4 gene (see Schumann et al., Science 249:1429-31 (1990)) was amplified by PCR from genomic DNA of a C57BL/6 mouse and cloned into the pGL3-basic vector (Promega). Constructions of the Tlr4 promoter were obtained using exonuclease III or restriction endonucleases. Sequence analysis of the −518 to −102 bp region of the Tlr4 promoter revealed the presence of two conserved PU.1-binding sites at postitions -292 to -286 and -115 to -109. Mutations confirmed by sequencing of the two PU.1-binding sites (from TTCCTCT (SEQ ID NO: 3) to TAGTTCT (SEQ ID NO: 4)) were obtained by PCR using the QuickChange site-directed mutagenesis kit (Stratagene).

### EXAMPLE 7

### NF-κB, TLR4 Promoter and PU.1 Luciferase Assays

Control and antisense MIF RAW 264.7 macrophages were incubated in 6-well tissue-culture plates and transiently transfected on the following day using Fugene6 transfection reagent (Roche Diagnostics) and 2 μg of a multimeric NF-κB pGL2 luciferase vector and 0.05 μg of the Renilla pRL-TK vector (Promega), together with 0.4-2 μg of the other expression constructs or with 2 μg of Tlr4 promoter pGL3 (see Rehli et al., J. Biol. Chem. 275:9773-81 (2000)) or an MHC-derived PU.1 (see Matsumura et al., Blood 96:2440-50 (2000)) luciferase vector and 0.04-2 μg of the Renilla pRL-TK vector (Promega). Twenty-four hours after transfection, cells were stimulated for 6 h with LPS (100 ng ml-⁻¹) and luciferase and Reni luciferase activities were measured using the Dual-Luciferase Reporter Assay System (Promega) Results are expressed as relative luciferase activity (ratio of luciferase/Reni luciferase activities).

### EXAMPLE 8

### Flow Cytometric Analysis

Binding of FITC-labeled Serratia marcescens LPS*LBP complexes onto RAW 264.7 macrophages was assessed by flow cytometry as described previously in Le Roy et al., J. Immunol. 162:7454-7460 (1999). After blocking Fc receptors with 2.4G2 hybridoma supernatant, expression of membrane-bound CD 14 and TLR4-MD-2 complex was evaluated by first incubating cells with a rat anti-mouse CD14 (see Adachi et al., J. Endotoxin Res. 5:139-46 (1999)) or TLR4-MD2 (see Akashi et al., J. Immunol. 164:3471-3475 (2000) or an isotype-matched control monoclonal antibody and then with a FITC-conjugated goat anti-rat antibody (Biosource International). Macrophages were counterstained with a phytorerythrin-conjugated anti-Mac I monoclonal antibody (Pharmingen). The amounts of TLR4-MD-2 on MIF^{+/+} and MIF^{-/-} macrophages are expressed as a fold increase of the mean fluorescence intensity over background.

### EXAMPLE 9

### Examination of Molecular Mechanism By Which MIF Regulates Responses of Macrophages to LPS and Gram-negative Bacteria

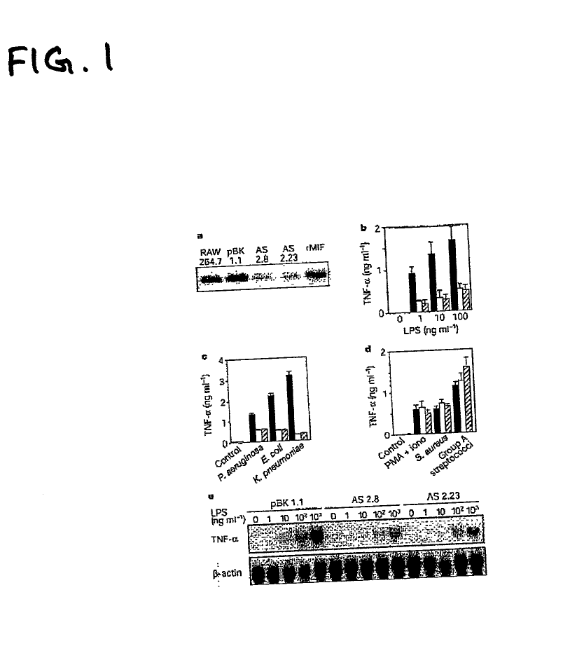

As MIF is highly expressed in the macrophage and given that it was recently shown to exert intracellular activity (see Kleemann et al., Nature 408:211-16 (2000)), it was hypothesized that reducing the endogenous MIF content might be a powerful research tool to address these questions. See Roger et al., Nature 414:920-24 (2001) (incorporated herein by reference in its entirety). Therefore, RAW 264.7 mouse macrophages were stably transfected with a plasmid encoding an antisense MIF mRNA. More than 20 clones were generated, two of which (designated as AS 2.8 and AS 2.23) were selected as they exhibited a marked reduction of MIF protein compared to control macrophages transfected with the empty plasmid (FIG. 1a). In contrast to control macrophages, antisense MIF macrophages were found to be hyporesponsive to endotoxin (Escherichia coli O111:B4 LPS) stimulation, as shown by a 3 to 12-fold reduction of TNF-α and IL-6 production (FIG. 1b). Similar results were obtained when antisense MIF macrophages were exposed to heat-killed Escherichia coli Klebsiella pneumoniae and Pseudomonas aeruginosa, the three most frequent causes of Gram-negative sepsis in humans (FIG. 1c). In contrast, control and antisense MIF macrophages responded equally well to stimulation with Gram-positive bacteria (group A streptococci, Staphylococcus aureus or Streptococcus mitis) or with phorbol 12-myristate 13-acetate plus calcium ionophore (FIG. 1d). Therefore, antisense MIF macrophages displayed a defect in the response to LPS and Gram-negative bacteria, but not to the other stimuli tested, suggesting a role for MIF in the signaling pathways activated by LPS in the macrophage.

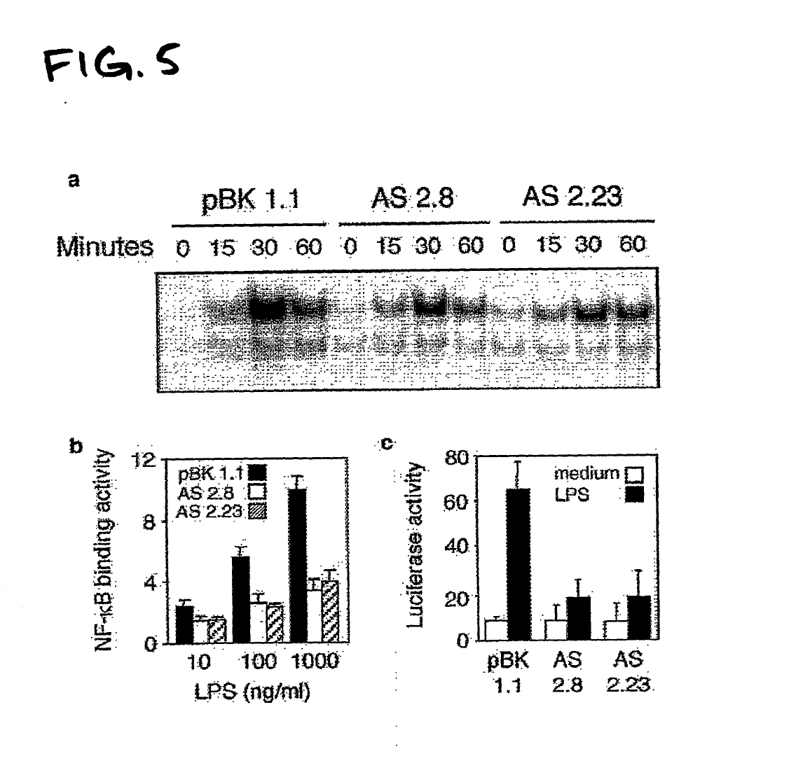

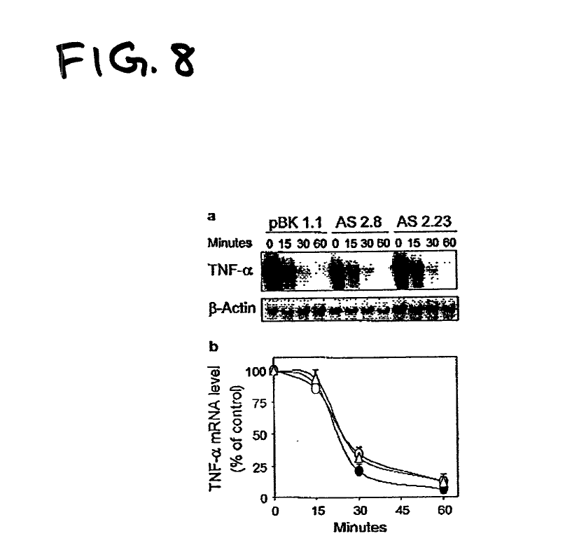

To explore the molecular mechanism(s) by which MIF regulates responses of macrophages to LPS and Gram-negative bacteria, we then examined the intracellular events controlling the expression of the TNF-α gene. Nuclear factor kappa B (NF-κB) DNA binding activity (FIG. 5a, b), NF-κB- driven luciferase reporter activity (FIG. 5c) and TNF-α: messenger RNA (mRNA) levels were all found to be markedly decreased in antisense MIF macrophages stimulated with LPS. The latter was not due to an increased turnover of TNF-α: mRNA. (See FIG. 8). These results suggested that reducing the endogenous level of MIF in the macrophage impedes LPS signal transduction at a point positioned between the binding of LPS to its receptor complex and the activation of NF-κB.

Recognition of LPS and Gram-negative bacteria by the host requires the cooperative interplay between the LPS-binding protein (LBP) (see Schumann et al., Science 249:1429-31 (1990)), CD14 (see Wright et al., Science 249:1431-1433 (1990)), and the recently identified Toll-like receptor 4 (TLR4) (see Poltorak et al., Science 282:2085-88 (1998)). LBP binds and transfers LPS-containing particles to a receptor complex composed of CD14, a ligand-binding GPI-anchored protein, and TLR4, the molecule that transduces the LPS signal. TLR4, the first identified human homologue of the Drosophila Toll receptor, was originally shown to activate NF-κB and to induce the expression of pro-inflammatory cytokines by human monocytic cells. See Medzhitov et al., Nature 388:394-97 (1997). Recently, Beutler and colleagues linked the defective LPS signaling of C3H/HeJ and C57BI/lOScCr mice to missense and null mutations of the Tlr4 gene, respectively. See Poltorak et al., Science 282:2085-88 (1998). This critical observation and a series of studies in mice and humans have unequivocally demonstrated that TLR4 is an essential component of the LPS receptor complex. See Chow, et al., J. Biol. Chem. 274:10689-92 (1999); Hoshino et al., J. Immunol. 162:3749-52 (1999); Heine et al., J. Immunol. 162:6971-75 (1999); Arbour et al., Nat. Genet. 25:187-191 (2000).

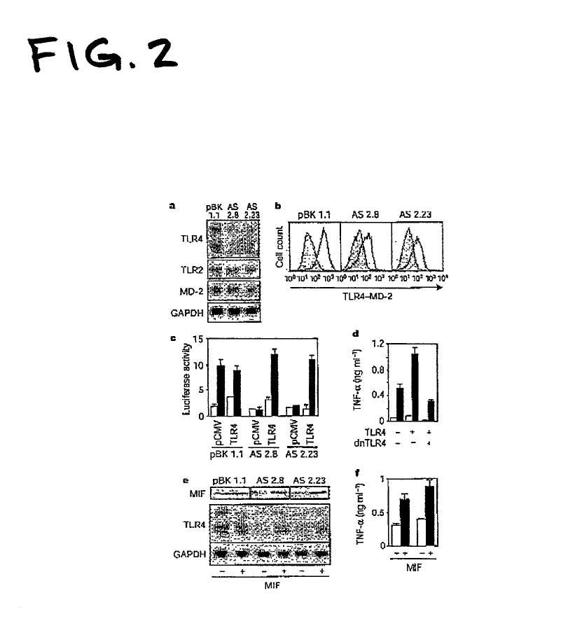

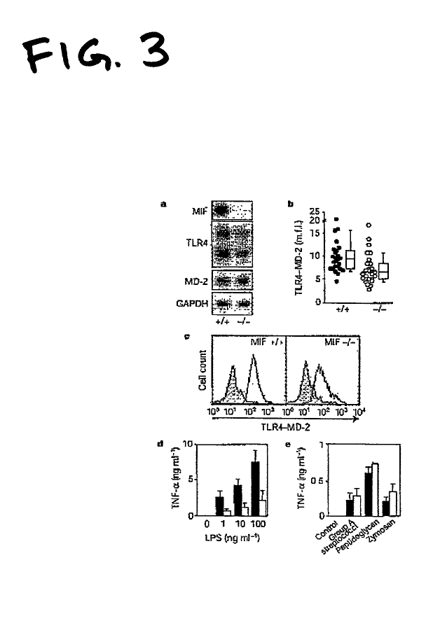

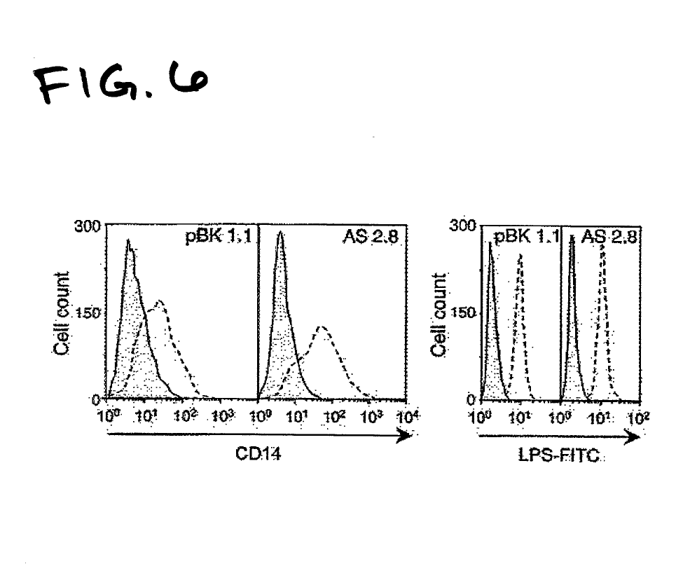

Therefore, it was investigated whether LBP, CD 14 or TLR4 were implicated in the impaired response of antisense MIF macrophages to LPS. Control and antisense macrophages expressed comparable levels of CD14 and bound complexes of FITC-labelled LPS-LBP equally well. (See FIG. 6). In contrast to CD14, baseline levels of TLR4 mRNA (FIG. 2a) and TLR4 protein (FIG. 2b) were considerably reduced in antisense MIF macrophages. Messenger RNA levels of MD-2, a secreted protein that associates with the extracellular domain of TLR4 and augments the cell response to LPS, were similar in control and antisense MIF macrophages. Unlike TLR4, the expression of TLR2 mRNA, a member of the TLR family mediating host responses to components of Gram-positive bacteria, mycobacteria and yeast (see Schwandner et al., J. Biol. Chem. 274:17406-409 (1999); Takeuchi et al., Immunity 11:443-51 (1999); Underhill et al., Nature 401:811-15 (1999)), was preserved in antisense MIF macrophages (FIG. 3a). Conservation of TLR2 expression is congruous with the observation that antisense MIF macrophages responded normally to Gram-positive bacteria (FIG. 1d).

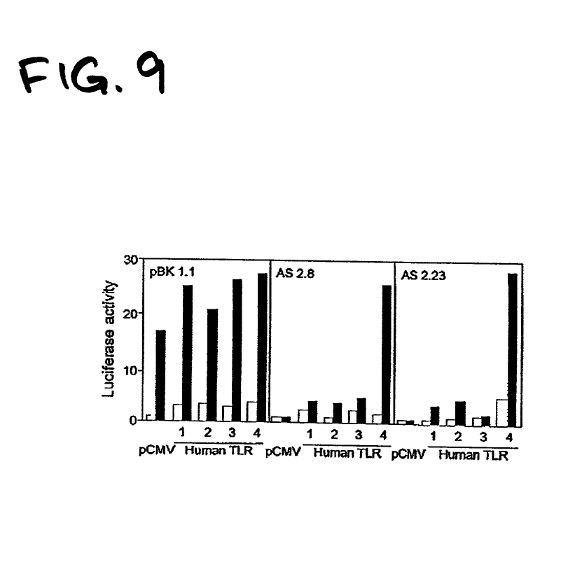

To substantiate the observation that the down-regulation of TLR4 accounted for the abnormal response of antisense MIF macrophages to LPS stimulation, cells were transfected with an expression vector encoding a mouse TLR4 cDNA. Adding back mouse TLR4 corrected the defective response to LPS, as shown by the restoration of NF-κB activity and TNF-α production (FIG. 2c, d). Similar results were obtained when antisense MIF macrophages were transfected with an expression vector encoding human TLR4, whereas cells transfected with human TLR1, TLR2 or TLR3 remained unresponsive to LPS. (See FIG. 9). Moreover, the restoration of a normal LPS signaling in antisense MIF macrophages transfected with mouse TLR4 was inhibited by co-expression of a dominant-negative mutant of mouse TLR4 with a deletion of 65 amino acids at the intracytoplasmic C-terminus of the molecule (FIG. 2d). Alternatively, the endogenous MIF protein content was reconstituted by transiently transfecting antisense MIF macrophages with an expression vector encoding a mouse MIF cDNA. Raising the intracellular MIF content of antisense MIF macrophages to levels similar to those in control macrophages restored both basal TLR4 mRNA expression and TNF-α production in cells stimulated with LPS (FIG. 2e, f). Taken together, these results strongly suggest that a reduction of endogenous MIF impairs the ability of macrophages to respond to LPS and Gram-negative bacteria via a down-regulation of TLR4.

Next, the importance of MIF in host responses to endotoxin was examined in vivo using macrophages from MIF knockout (MIF^{-/-}) mice. See Bozza et al., J. Exp. Med. 189:341-46 (1999). Resident and thioglycollate-elicited peritoneal macrophages were isolated from MIF^{-/-} and MIF^{+/+} mice. Like antisense MIF macrophages, peritoneal MIF macrophages were found to exhibit a substantial down-regulation of TLR4 protein as assessed by flow cytometry (FIG. 3b). MIF macrophages were also hyporesponsive to stimulation with LPS, as manifested by a 3-fold reduction of TNF-α output at all LPS concentrations tested (FIG. 3d). Similar to MIF mice, mice treated with anti-MIF antibodies have been shown to be protected from lethal endotoxemia and Gram-negative septic shock. See Bernhagen et al., Nature 365:756-59 (1993); Calandra et al., Nature 377:68-71 (1995); Calandra et al., Nat. Med. 6:164-170 (2000); Bozza et al., J. Exp. Med. 189:341-46 (1999). Of note, increased survival was associated with a reduction of the systemic TNF-α concentrations. TLR4 expression and TNF-α production were also significantly decreased in macrophages treated with anti-MIF antibodies.

Thus, deletion of the Mif gene or neutralization of MIF activity down-regulates TLR4 expression protecting the macrophage from being overstimulated by LPS, as it may occur during severe Gram-negative sepsis. Such overstimulation would otherwise result in overwhelming cytokine release, leading to tissue injury, multiple organ dysfunction, shock and death. These results identify an important part for MIF in the early phase of sepsis and provide the molecular basis for a mechanism by which MIF mice and mice treated with MIF antibodies may be protected from lethal endotoxemia and Gram-negative septic shock. However, given MIF's powerful pro-inflammatory properties (see Calandra et al., J. Exp. Med. 179:1895-902 (1994); Bacher et al., Proc. Natl. Acad. Sci. USA 93:7849-54 (1996)), it may also contribute to lethality by acting at a later stage of the cascade of events induced by sepsis.

These results strongly argue in favor of an essential role for MIF in host defenses against Gram-negative sepsis. Moreover, they also provide a rationale for the intriguing observation that MIF is constitutively expressed by macrophages and by tissues in direct contact with the environment. See Calandra et al., J. Exp. Med. 179:1895-902 (1994); Bacher et al., Amer. J. Pathol. 150:235-46 (1997)). Recognition of invasive microbial pathogens is a critical function of innate immunity. By up-regulating the basal expression of TLR4 in the macrophage, MIF promotes the recognition of endotoxin-containing particles and Gram-negative bacteria by the innate immune system. Thereby, MIF enhances the production of inflammatory cytokines, such as TNF-α, and facilitates the mounting of the host defensive response.

In support of MIF's critical part in innate host defenses, it has been observed that MIF-deficient macrophages exhibit a cytokine-dependent impaired capacity to kill Gram-negative bacteria (such as E. coli and K. pneumoniae), which can be corrected by the addition of exogenous TNF-α(data not shown). Therefore, MIF enables cells that are at the front-line of antimicrobial host defenses, like the macrophage, to respond quickly to hostile Gram-negative bacteria. Thus, these findings identify a critical role for MIF in innate immunity and provides a rationale for anti-MIF treatment strategy in patients with Gram-negative septic shock.

### Other Embodiments

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method for treating conditions involving a mediator-induced diseases or pathology comprising administering a therapeutically effective amount of an agent that decreases the endogenous amount of intracellular or extracellular MIF to a patient.

2. The method of claim 1, wherein the condition involving mediator-induced diseases or pathology is selected from the group consisting of endotoxin-induced septic shock, endotoxin-induced toxic shock, sepsis, severe sepsis, septic shock caused by Gram-negative bacteria, bacterial infections, shock, inflammatory diseases, graft versus host disease, autoimmune diseases, acute respiratory distress syndrome, granulomatous diseases, chronic infections, transplant rejection, acute respiratory asthma, viral infections, parasitic infections, fungal infections, and trauma.

3. The method of claim 1, wherein the agent is selected from the group consisting of an antisense nucleic acid, a small molecule inhibitory compound, a peptide mimetic, an agent that down-regulates MIF expression, an antibody, and an inhibitor of MIF activity.

4. The method of claim 2, wherein the mediator-induced diseases or pathology is the result of infectious agents.

5. A method for treating a disease selected from the group consisting of graft versus host disease, acute respiratory distress syndrome, granulomatous diseases, transplant rejection, cachexia, parasitic infections, fungal infections, trauma, and bacterial infections by administering a therapeutically effective amount of an agent that decreases the endogenous amount of intracellular or extracellular MIF to a patient.

6. The method of claim 5, wherein the agent is selected from the group consisting of an antisense nucleic acid, a small molecule inhibitory compound, a peptide mimetic, an agent that down-regulates MIF expression, an antibody, and an inhibitor of MIF activity.

7. The method of claim 5, wherein the disease is the result of infectious agents.

8. A method for treating an inflammatory or infectious condition or disease comprising administering a therapeutically effective amount of an agent that decreases the endogenous amount of intracellular or extracellular MIF to a patient.

9. The method of claim 8, wherein the agent is selected from the group consisting of an antisense nucleic acid, a small molecule inhibitory compound, a peptide mimetic, an agent that down-regulates MIF expression, an antibody, and an inhibitor of MIF activity.

10. The method of claim 8, wherein the inflammatory or infectious condition or disease is the result of infectious agents.

11. A method for treating an individual having a disease caused by a mediator-induced diseases or pathology comprising administering to the individual an effective amount of an agent that decreases the endogenous amount of intracellular or extracellular MIF and a pharmaceutically acceptable carrier or diluent.

12. The method of claim 11, wherein the disease caused by a mediator-induced diseases or pathology is selected from the group consisting of endotoxin-induced septic shock, endotoxin-induced toxic shock, sepsis, severe sepsis, septic shock caused by Gram-negative bacteria, bacterial infections, shock, inflammatory diseases, graft versus host disease, autoimmune diseases, acute respiratory distress syndrome, granulomatous diseases, chronic infections, transplant rejection, acute respiratory asthma, viral infections, parasitic infections, fungal infections, and trauma.

13. The method of claim 11, wherein the agent is selected from the group consisting of an antisense nucleic acid, a small molecule inhibitory compound, a peptide mimetic, an agent that down-regulates MIF expression, an antibody, and an inhibitor of MIF activity.

14. The method of claim 12, wherein the disease caused by a mediator-induced diseases or pathology is the result of infectious agents.

15. The method of claim 11 further comprising administering a therapeutic steroid.

16. A method for enhancing the anti-inflammatory activity of a therapeutic steroid or reducing the toxic side effects of the therapeutic steroid comprising administering a therapeutically effective amount of an agent that decreases the endogenous amount of intracellular or extracellular MIF to an individual.

17. The method of claim 16, wherein the agent is selected from the group consisting of an antisense nucleic acid, a small molecule inhibitory compound, a peptide mimetic, an agent that down-regulates MIF expression, an antibody, and an inhibitor of MIF activity.

18. A method for treating a condition involving a mediator-induced diseases or pathology comprising administering to a patient an effective amount of an agent that down-regulates a Toll-like receptor.

19. The method of claim 18, wherein the Toll-like receptor is Toll-like receptor 4.

20. The method of claim 18, wherein the condition involving a mediator-induced diseases or pathology is selected from the group consisting of endotoxin-induced septic shock, endotoxin-induced toxic shock, sepsis, severe sepsis, septic shock caused by Gram-negative bacteria, bacterial infections, shock, inflammatory diseases, graft versus host disease, autoimmune diseases, acute respiratory distress syndrome, granulomatous diseases, chronic infections, transplant rejection, acute respiratory asthma, viral infections, parasitic infections, fungal infections, and trauma.

21. The method of claim 18, wherein the agent that down-regulates the Toll-like receptor decreases the endogenous amount of intracellular or extracellular MIF.

22. The method of claim 21, wherein the agent is selected from the group consisting of an antisense nucleic acid, a small molecule inhibitory compound, a peptide mimetic, an agent that down-regulates MIF expression, an antibody, and an inhibitor of MIF activity.

23. The method of claim 20, wherein the condition involving a mediator-induced diseases or pathology is the result of infectious agents.

24. The method of claim 18, wherein the agent is selected from the group consisting of an antisense nucleic acid, a small molecule inhibitory compound, a peptide mimetic, an agent that down-regulates Toll-like receptor expression, and an inhibitor of Toll-like receptor activity.

25. The method of claim 19, wherein the agent is selected from the group consisting of an antisense nucleic acid, a small molecule inhibitory compound, a peptide mimetic, an agent that down-regulates Toll-like Receptor 4 expression, and an inhibitor of Toll-like Receptor 4 activity

26. A method for treating a condition involving a mediator-induced diseases or pathology comprising administering a therapeutically effective amount of an anti-MIF antibody or antigen binding fragment thereof in combination with an agent that decreases the endogenous amount of intracellular or extracellular MIF to a patient.

27. The method of claim 26, wherein the condition involving a mediator-induced diseases or pathology is selected from the group consisting of endotoxin-induced septic shock, endotoxin-induced toxic shock, sepsis, severe sepsis, septic shock caused by Gram-negative bacteria, bacterial infections, shock, inflammatory diseases, graft versus host disease, autoimmune diseases, acute respiratory distress syndrome, granulomatous diseases, chronic infections, transplant rejection, acute respiratory asthma, viral infections, parasitic infections, fungal infections, and trauma.

28. The method of claim 26, wherein the agent is selected from the group consisting of an antisense nucleic acid, a small molecule inhibitory compound, a peptide mimetic, an agent that down-regulates MIF expression, an antibody, and an inhibitor of MIF activity.

29. The method of claim 27, wherein the condition involving a mediator-induced diseases or pathology is the result of infectious agents.
